SEQUENCE LISTING (28 sequences)

SEQ ID NO: 1 - protein, 113 aa, synthetic construct
DIVMTQSPDSLAVSLGERATINCKSSQSVLRSSNNENYLAWYQQKPGQPPKLLIYGASTRESGVPDRFSGSGSGTDFTLTISSLQAEDVAVYYCQQWYSAPWTFGQGTKVEIK

SEQ ID NO: 2 - protein, 120 aa, synthetic construct
EVQLLESGGGLVQPGGSLRLSCAASGFTFSSYAMSWVRQAPGKGLEWVSGISGYGGDTDYADSVKGRFTISRDNSKNTLYLQMNSLRAEDTAVYYCAGPGYCSGGSCYRWGQGTLVTVSS

SEQ ID NO: 3 - protein, 113 aa, synthetic construct
DIVMTQSPDSLAVSLGERATINCKSSQSVLYSSNNKNYLAWYQQKPGQPPKLLIYAASTRESGVPDRFSGSGSGTDFTLTISSLQAEDVAVYYCQQWYRAPYTFGQGTKVEIK

SEQ ID NO: 4 - protein, 122 aa, synthetic construct
EVQLLESGGGLVQPGGSLRLSCAASGFTFSSYAMSWVRQAPGKGLEWVSAISGSGYGGDTYYADSVKGRFTISRDNSKNTLYLQMNSLRAEDTAVYYCAGPGYCSGGSCYRWGQGTLVTVSS

SEQ ID NO: 5 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQHESWPITFGQGTKVEIK

SEQ ID NO: 6 - protein, 112 aa, synthetic construct
EVQLLESGGGLVQPGGSLRLSCAASGFTFTNYAMSWVRQAPGKGLEWVSAISGSGGSTSYADSVKGRFTISRDNSKNTLYLQMNSLRAEDTAVYYCARWDYWGQGTLVTVSS

SEQ ID NO: 7 - protein, 113 aa, synthetic construct
DIVMTQSPDSLAVSLGERATINCKSSQSVLYSSNNENYLAWYQQKPGQPPKLLIYGASTRESGVPDRFSGSGSGTDFTLTISSLQAEDVAVYYCQQGSNAPYTFGQGTKVEIK

SEQ ID NO: 8 - protein, 119 aa, synthetic construct
EVQLLESGGGLVQPGGSLRLSCAASGFTFTDYQMSWVRQAPGKGLEWVSAIDGSDTYYADSVKGRFTISRDNSKNTMYLQMNSLRAEDKAVYYCARAPSGWAPGYFDYWGQGTLVTVSS

SEQ ID NO: 9 - protein, 113 aa, synthetic construct; identical to SEQ ID NO: 7

SEQ ID NO: 10 - protein, 119 aa, synthetic construct
EVQLLESGGGLVQPGGSLRLSCAASGFTFTDYQMSWVRQAPGKGLEWVSAIDGSDTYYADSVKGRFTISRDNSKNTLYLQMNSLRAEDTAVYYCARASSGWAPGYFDYWGQGTLVTVSS

SEQ ID NO: 11 - protein, 113 aa, synthetic construct
DIVMTQSPDSLAVSLGERATINCKSSQSVLRSNNNENYLAWYQQKPGQPPKLLIYGASTRESGVPDRFSGSGSGTDFTLTISSLQAEDVAVYYCQQGYRYPLTFGQGTKVEIK

SEQ ID NO: 12 - protein, 123 aa, synthetic construct
EVQLLESGGGLVQPGGSLRLSCAASGFTFSDYAMSWVRQAPGKGLEWVSAISGGGGYTYYADSVKGRFTISRDNSKNTLYLQMNSLRAEDTAVYYCAKNRCSDYCIPDWFDPWGQGTLVTVSS

SEQ ID NO: 13 - protein, 113 aa, synthetic construct
DIVMTQSPDSLAVSLGERATINCKSSQSVLYSDNNENYLAWYQQKPGQPPKLLIYWASTRESGVPDRFSGSGSGTDFTLTISSLQAEDVAVYYCQQWYSEPFTFGQGTKVEIK

SEQ ID NO: 14 - protein, 120 aa, synthetic construct
EVQLLESGGGLVQPGGSLRLSCAASGFTFTDDAMSWVRQAPAKGLEWVSAISGRGGTTNYADSVKGRFTISRDNSNNTLYLQMNSLRAEDTAVYYCAGPGYCSGGSCYRWGQGTLVTVSS

SEQ ID NO: 15 - protein, 108 aa, synthetic construct
EIVLTQSPGTLSLSPGERATLSCRASQSVSASYLAWYQQKPGQAPRLLIYGASSRATGIPDRFSGSGSGTDFTLTISRLEPEDFAVYYCQQYSSYPFTFGQGTKVEIK

SEQ ID NO: 16 - protein, 112 aa, synthetic construct
EVQLLESGGGLVQPGGSLRLSCAASGFTFTSYGMSWVRQAPGKGLEWVSGISGSGYTTSYADSVKGRFTISRDNSKNTLYLQMNSLRAEDTAVYYCASGGYWGQGTLVTVSS

SEQ ID NO: 17 - protein, 108 aa, synthetic construct
EIVLTQSPGTLSLSPGERATLSCRASQSVSSSALAWYQQKPGQAPRLLIYGASSRATGIPDRFSGSGSGTDFTLTISRLEPEDFAVYYCQQHGSSPYTFGQGTKVEIK

SEQ ID NO: 18 - protein, 122 aa, synthetic construct
EVQLLESGGGLVQPGGSLRLSCAASGFTFTDSGMSWVRQAPGKGLEWVSRISGRGGTTDYADSVKGRFTISRDNSKNTLYLQMNSLRAEDTAVYYCATDPVFGYSSGWRDYWGQGTLVTVSS

SEQ ID NO: 19 - protein, 108 aa, synthetic construct
EIVLTQSPGTLSLSPGERATLSCRASQSVSASNLAWYQQKPGQAPRLLIYAASSRATGIPDRFSGSGSGTDFTLTISRLEPEDFAVYYCQQHGNSPFTFGQGTKVEIK

SEQ ID NO: 20 - protein, 116 aa, synthetic construct
EVQLLESGGGLVQPGGSLRLSCAASGFTFSDSGMSWVRQAPGKGLEWVSRIYGSDTYYADSVKGRFTISRDNSKNTLYLQMNSLRAEDTAVYYCATDPFYSYVDYWGQGTLVTVSS

SEQ ID NO: 21 - protein, 113 aa, synthetic construct
DIVMTQSPDSLAVSLGERATINCKSSQSVLYSDNNENRLAWYQQKPGQPPKLLIYAASTRESGVPDRFSGSGSGTDFTLTISSLQAEDVAVYYCQQWTSAPYTFGQGTKVEIK

SEQ ID NO: 22 - protein, 122 aa, synthetic construct
EVQLLESGGGLVQPGGSLRLSCAASGFTFSSYAMSWVRQAPGKGLEWVSAISGSGYGGSTSYADSVKGRFTISRDNSKNTLYLQMNSLRAEDTAVYYCAGPGYCSGGSCYRWGQGTLVTVSS

SEQ ID NO: 23 - protein, 113 aa, synthetic construct
DIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYYNSPITFGQGTKVEIK

SEQ ID NO: 24 - protein, 126 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHWTYYFGSRSYSGAFDIWGQGTMVTVSS

SEQ ID NO: 25 - protein, 113 aa, synthetic construct
DIVMTQSPDSLAVSLGERATINCKSSQSVLYSSNNENRLAWYQQKPGQPPKLLIYGASTRESGVPDRFSGSGSGTDFTLTISSLQAEDVAVYYCQQYSNSPITFGQGTKVEIK

SEQ ID NO: 26 - protein, 126 aa, synthetic construct
EVQLLESGGGLVQPGGSLRLSCAASGFTFSDYGMSWVRQAPGKGLEWVSGISGRGGGTRYADSVKGRFTISRDNSKNTLYLQMNSLRAEDTAVYYCARGVTHCTNGVCHYWYFDLWGRGTLVTVSS

SEQ ID NO: 27 - protein, 108 aa, synthetic construct
EIVLTQSPGTLSLSPGERATLSCRASQSVSSSALAWYQQKPGQAPRLLIYGASSRATGIPDRFSGSGSGTDFTLTISRLEPEDFAVYYCQQHSSSPFTFGQGTKVEIK

SEQ ID NO: 28 - protein, 124 aa, synthetic construct
EVQLLESGGGLVQPGGSLRLSCAASGFTFSSYGMSWVRQAPGKGLEWVSRISGSGDGSYTNYADSVKGRFTISRDNSKNTLYLQMNSLRAEDTAVYYCASDPITMIVGDAFDIWGQGTMVTVSS

(19) United States
(12) Patent Application Publication
(10) Pub. No.: US 2026/0049981 A1
(43) Pub. Date: Feb. 19, 2026

(54) ENRICHMENT OF PARTICLE REAGENTS FOR ARRAY-BASED ASSAYS

(71) Applicant: Nautilus Subsidiary, Inc., Seattle, WA (US)

(72) Inventors: Zhengjian ZHANG, Albany, CA (US); Maureen NEWMAN, Belmont, CA (US); Harman SINGH, South San Francisco, CA (US); Brooks HOEHN, Palo Alto, CA (US); Scott Michael TABAKMAN, San Diego, CA (US); Vivekananda BUDAMAGUNTA, San Carlos, CA (US)

(21) Appl. No.: 19/249,855

(22) Filed: Jun. 25, 2025

Related U.S. Application Data
(60) Provisional application No. 63/664,889, filed on Jun. 27, 2024.

(51) Int. Cl. G01N 33/543

(52) U.S. Cl. CPC G01N 33/54306; G01N 33/54326; G01N 33/54386; G01N 2440/00

(57) ABSTRACT

Methods of separating fully-formed particles from particle mixtures containing partially-formed particles are set forth. Fully-formed particles and partially-formed particles may be bound to a solid support, followed by selective dissociation of the fully-formed particles from the solid support, thereby providing a particle mixture that is enriched for the fully-formed particles.

### CROSS-REFERENCE TO RELATED APPLICATIONS

This application claims priority to U.S. Provisional Application No. 63/664,889, filed on Jun. 27, 2024, the content of which is incorporated herein by reference in its entirety.

### SEQUENCE LISTING

This application contains a Sequence Listing which has been submitted electronically in XML format and is hereby incorporated by reference in its entirety. Said XML copy, created on Jun. 20, 2025, is named “SL_50109_4026WO.xml” and is 41,622,941 bytes in size.

### BACKGROUND

Methods of preparing reagents for chemical or physical processes may inherently produce a diversity of products, including intended products and unintended products, such as intermediate products, side products, and mis-formed products. After forming a diversity of products, the products may be fractionated or otherwise purified to remove unintended products or enrich for intended products.

It may be useful to prepare reagents by complexing molecules such as proteins or other biomolecules to particles or macromolecules. Molecules may be attached to the particles or macromolecules by attachment handles that facilitate coupling. Accordingly, after a process of complexing molecules to particles or macromolecules, unintended products of the attachment may include particles or macromolecules with unattached attachment handles, as well as particles or macromolecules with no attachment handles and unattached molecules. When introduced into assays or other downstream processes, the unintended macromolecular products may produce deleterious or suboptimal results. Accordingly, removal of unintended products of macromolecular complex formation may improve assays or other downstream processes that utilize the macromolecular complexes.

### SUMMARY

The present disclosure provides a method for separating particles. The method can include the steps of: (a) contacting in a fluid phase a plurality of particles to a solid support, wherein the plurality of particles comprises: (i) a first set of particles, wherein each particle of the first set of particles comprises a first attachment handle, and (ii) a second set of particles, wherein each particle of the second set of particles comprises a second attachment handle, and wherein each particle of the second set of particles is attached to an entity, (b) attaching first attachment handles of particles of the first set of particles or second attachment handles of particles of the second set of particles to the solid support, and (c) after attaching the first set of particles or particles of the second set of particles to the solid support, separating the first set of particles from the second set of particles.

In some embodiments the method may further comprise a step of attaching entities to particles, thereby forming the second set of particles. In some configurations, the entity of the entities can comprise a plurality of second attachment handles. In some cases, attaching entities to particles can comprise attaching a second attachment handle of the plurality of second attachment handles of the entity to a first attachment handle of a particle of the particles. In other cases, attaching entities to particles can comprise attaching a first coupling moiety of a particle of the particles to a complementary coupling moiety of an entity of the entities. In some configurations, a first attachment handle can be attached to the coupling moiety of the particle of the particles, wherein attaching the first coupling moiety of the particle of the particles to the complementary coupling moiety of the entity of the entities further can comprise dissociating the first attachment handle from the coupling moiety of the particle of the particles.

In some embodiments, separating the first set of particles from the second set of particles can comprise separating the fluid phase from the solid support. In some cases, the method may further comprise, after separating the fluid phase from the solid support, dissociating particles from the solid support.

In some embodiments, attaching first attachment handles of particles of the first set of particles or second attachment handles of particles of the second set of particles to the solid support can comprise attaching first attachment handles of particles of the first set of particles to complementary attachment handles of the solid support. In some cases, attaching first attachment handles of particles of the first set of particles to the complementary attachment handles of the solid support can comprise forming dissociable binding interactions between first attachment handles of particles of the first set of particles and the complementary attachment handles of the solid support. In other cases, attaching first attachment handles of particles of the first set of particles to the complementary attachment handles of the solid support can comprise forming non-dissociable binding interactions between first attachment handles of particles of the first set of particles and the complementary attachment handles of the solid support.

In some embodiments, attaching first attachment handles of particles of the first set of particles or second attachment handles of particles of the second set of particles to the solid support can comprise attaching first attachment handles of particles of the first set of particles and second attachment handles of particles of the second set of particles to complementary attachment handles of the solid support. In some cases, attaching first attachment handles of particles of the first set of particles and second attachment handles of particles of the second set of particles to complementary attachment handles of the solid support can comprise forming non-dissociable binding interactions between first attachment handles of the particles of the first set of particles and the complementary attachment handles of the solid support, and can further comprise forming dissociable binding interactions between second attachment handles of the particles of the second set of particles and the complementary attachment handles of the solid support.

In some embodiments, attaching first attachment handles of particles of the first set of particles or second attachment handles of particles of the second set of particles to the solid support can comprise coupling the first attachment handles or the second attachment handles to affinity reagents. In some configurations, the affinity reagents can be immobilized on the solid support. In some cases, coupling the first attachment handles or the second attachment handles to affinity reagents can comprise incubating the affinity reagents with the first attachment handles or the second attachment handles in the fluid phase. In some cases, the method may further comprise, after incubating the affinity reagents with the first attachment handles or the second attachment handles in the fluid phase, attaching the affinity reagents bound to first attachment handles or second attachment handles to the solid support. In some cases, attaching the affinity reagents bound to first attachment handles or second attachment handles to the solid support comprises attaching a third attachment handle of an affinity reagent to a complementary attachment handle of the solid support.

The present disclosure provides another method for separating particles. The method can include the steps of: (a) contacting in a fluid phase a plurality of particles to a solid support, wherein the solid support comprises a plurality of complementary attachment handles, and wherein the plurality of particles comprises: (i) a first set of particles, wherein the first set of particles is attached to a plurality of polypeptides, wherein each polypeptide of the plurality of polypeptides comprises a plurality of attachment handles, and (ii) a second set of particles, wherein each particle of the second set of particles is not attached to a polypeptide, (b) attaching attachment handles of polypeptides to the plurality of complementary attachment handles of the solid support, thereby attaching particles of the first set of particles to the solid support, and (c) after attaching particles of the first set of particles to the solid support, separating the particles of the first set of particles from the second set of particles.

In some embodiments, the method may further comprise a step of for each polypeptide of the plurality of polypeptides forming the plurality of attachment handles. In some cases, forming the plurality of attachment handles on a polypeptide of the plurality of polypeptides can comprise attaching an exogenous moiety to the polypeptide of the plurality of polypeptides. In other cases, forming the plurality of attachment handles on a polypeptide of the plurality of polypeptides can comprise attaching an endogenous moiety to the polypeptide of the plurality of polypeptides.

The present disclosure provides another method for separating particles. The method can include the steps of: (a) contacting in a fluid phase a plurality of particles to a solid support, wherein the solid support comprises a plurality of complementary first attachment handles, and wherein the plurality of particles comprises: (i) a first set of particles, wherein the first set of particles is attached to a plurality of polypeptides, wherein each polypeptide of the plurality of polypeptides is attached to a particle of the first set of particles by coupling of a second attachment handle of the polypeptide to a complementary second attachment handle of the particle, and (ii) a second set of particles, wherein each particle of the second set of particles is not attached to a polypeptide, and wherein each particle of the second set of particles comprising an unbound complementary second attachment handle, (b) attaching unbound complementary second attachment handles of particles of the second set of particles to the plurality of complementary attachment handles of the solid support, thereby attaching particles of the second set of particles to the solid support, and (c) after attaching particles of the second set of particles to the solid support, separating the particles of the first set of particles from the second set of particles.

The present disclosure further provides systems for particle separation. A system can comprise: (a) a vessel containing a fluid phase, wherein the fluid phase is contacted to a solid support within the vessel, (b) a particle mixture comprising a first set of particles and a second set of particles, (c) a particle transfer device, wherein the particle transfer device is configured to transfer the particle mixture to the vessel containing the fluid phase, and (d) a separation device, wherein the separation device is configured to separate the fluid phase from contact with the solid support.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A, 1B, 1C, 1D, 1E, 1F, and 1G depict steps of a method of enriching a particle mixture, in accordance with some embodiments.

FIGS. 2A, 2B, 2C, and 2D illustrate configurations of particles that are configured to be attached to analytes, in accordance with some embodiments.

FIGS. 3A, 3B, and 3C show configurations of particles that are configured to be attached to affinity agents and/or detectable labels, in accordance with some embodiments.

FIG. 4 displays a flow chart of a method for enriching a particle mixture, in accordance with some embodiments.

FIG. 5A depicts a method for attaching an analyte-linked particle to a solid support, in accordance with some embodiments.

FIGS. 5B, 5C, 5D, and 5E depict steps of a method of enriching a particle mixture, in accordance with some embodiments.

FIGS. 6A and 6B illustrate steps of a method of enriching a particle mixture, in accordance with some embodiments.

FIG. 7 shows an example of enrichment of a particle mixture in which the particles contain a dispersity of forms, in accordance with some embodiments.

FIG. 8 displays a system configured to perform certain methods that utilize enriched particle mixtures, as set forth herein, in accordance with some embodiments.

FIGS. 9A, 9B, and 9C depict steps of a method of forming an enriched particle mixture from a heterogeneous particle mixture, in accordance with some embodiments.

### DETAILED DESCRIPTION

Macromolecular complexes may be useful reagents for numerous processes. For example, nanoparticles, such as nucleic acid nanoparticles, may be useful for facilitating the orderly attachment of analytes (e.g., proteins, nucleic acids, polysaccharides, etc.) to surfaces. In another example, nanoparticles may be useful structures for attaching affinity agents and/or detectable labels to form multivalent binding reagents. In some cases, macromolecular binding reagents may be utilized to facilitate analysis of analytes attached via macromolecules to a solid support.

When molecules are attached to macromolecules, the attachment process is likely to result in unintended products. In particular, the attachment process may produce macromolecules comprising unutilized attachment handles. For example, macromolecules may be provided with a reactive functional group that can be utilized to covalently attach a molecule to the macromolecule. If the reactive functional group of a macromolecule is not reacted with a complementary reactive group of a molecule during formation of macromolecular complexes, the product mixture will include macromolecules containing unattached reactive functional groups. Unutilized attachment handles may be deleterious when reagents containing the unutilized attachment handles are utilized. For example, macromolecules with unutilized attachment handles may bind with other molecules in an unwanted or unexpected manner. Likewise, macromolecules with unutilized attachment handles may aggregate with other molecules or macromolecules, thereby forming unwanted aggregate products.

When complexed particles are formed by the attachment of an entity (e.g., an analyte, an affinity reagent, a tether strand, a detectable label, a receptor-ligand binding pair component) to a macromolecule or particle, a product mixture may be expected to comprise complexed particles, unattached macromolecules or particles, and unattached entities. The presence of unattached attachment handles on some constituents of a particle mixture may be useful for the separation of unformed, partially-formed, or over-formed constituents from the particle mixture. Recognized herein is the use of structural differences between properly-formed complexed particles and unformed, partially-formed, or over-formed complexed particles to effectuate a separation of the desired properly-formed complexed particles from the undesired complexed particles. In some cases, presence of attachment handles only in properly-formed complexed particles may facilitate separation of the properly-formed complexed particles from unformed, partially-formed, or over-formed complexed particles. Alternatively, presence of attachment handles only in unformed, partially-formed, or over-formed complexed particles may facilitate separation of the properly-formed complexed particles from properly-formed complexed particles. In particular, it may be useful to form complexed particles from macromolecules or particles comprising two differing types of attachment handles: 1) a dissociable attachment handle, and 2) a non-dissociable attachment handle.

Provided herein are methods of separating macromolecules and other unbound moieties from macromolecular complexes. Further provided herein are systems for separating macromolecules and other unbound moieties from macromolecular complexes. Further provided herein are methods for utilizing the macromolecular complexes in assays or other processes.

### Definitions

Terms used herein will be understood to take on their ordinary meaning in the relevant art unless specified otherwise. Several terms used herein and their meanings are set forth below.

As used herein, the term “binding reagent” refers to a composition comprising an affinity agent coupled to a detectable label. A binding reagent may comprise two or more affinity agents. A binding reagent may comprise two or more detectable labels. Optionally, a binding reagent may comprise a plasmonic particle system, as set forth herein. A binding reagent may comprise a particle (e.g., a nucleic acid nanoparticle, a polymer nanoparticle, a branched or dendrimeric nanoparticle) that couples one or more affinity agents to one or more detectable labels.

As used herein, the term “address” refers to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity agent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the terms “analyte” and “analyte of interest,” when used in reference to a structured nucleic acid particle, refer to a molecule, particle, or complex of molecules or particles that is coupled to a display moiety of a structured nucleic acid particle. An analyte may comprise a target for an analytical method (e.g., sequencing, identification, quantification, etc.) or may comprise a functional element such as a binding ligand or a catalyst. An analyte may comprise a biomolecule, such as a polypeptide, polysaccharide, nucleic acid, lipid, metabolite, enzyme cofactor or a combination thereof. An analyte may comprise a non-biological molecule, such as a polymer, metal, metal oxide, ceramic, semiconductor, mineral, or a combination thereof. As used herein, the terms “sample analyte” refers to an analyte derived from a sample collected from a biological or non-biological system. A sample analyte may be purified or unpurified. As used herein, the term “control analyte” refers to an analyte that is provided as a positive or negative control for comparison to a sample analyte. A control analyte may be derived from the same source as a sample analyte, or derived from a differing source from the sample analyte. As used herein, the term “standard analyte” refers to a known or characterized analyte that is provided as a physical or chemical reference to a process. A standard analyte may comprise the same type of analyte as a sample analyte, or may differ from a sample analyte. For example, a polypeptide analyte process may utilize a polypeptide standard analyte with known characteristics. In another example, a polypeptide analyte process may utilize a non-polypeptide standard analyte with known characteristics. As used herein, the term “inert analyte” refers to an analyte with no expected function in a process or system.

As used herein, the term “array” refers to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “attachment handle,” when used in reference to a particle, refers to a functional group, molecule, or moiety that facilitates attachment of the particle to an entity (e.g., an analyte, affinity agent, detectable label, a solid support, docker strand, or tether strand). An attachment handle may be configured to form a covalent or non-covalent interaction with a complementary attachment handle of an entity. Examples of non-covalent attachment handles can include oligonucleotides, members of a non-covalent receptor-ligand binding pair (e.g., streptavidin-biotin, avidin-biotin), members of affinity agent-antigen binding pairs (e.g., antibody-epitope, aptamer-epitope), and members of a chelating complex (e.g., nickel nitrilotriacetic acid (Ni-NTA) chelated to polyhistidine moieties). Examples of covalent attachment handles can include Click-type reagents and members of a covalent receptor-ligand binding pair (e.g., SpyCatcher-SpyTag, SnoopCatcher-SnoopTag, SdyCatcher-SdyTag). Attachment moieties associated with analytes may be endogenous relative to a system or organism from which the analytes are derived. For example, human proteins may comprise natural post-translational modifications (PTMs) that can be utilized as endogenous attachment handles, such as ubiquitins, phosphorylated residues, methylated residues, glycosylated residues, and acetylated terminal amino acids. Attachment moieties associated with analytes may be exogenous relative to a system or organism from which the analytes are derived. For example, human proteins may be modified with attachment handles comprising Click-type reagents, oligonucleotides, peptide tags containing synthetic, engineered, or non-human amino acid sequences, or molecules that are not naturally attached to human proteins.

As used herein, the term “complete complement,” when used in reference to a particle, refers to the particle being attached to an intended quantity of entities, such as analytes, affinity agents, detectable labels, docker strands, or tether strands, as set forth herein. For example, if a particle is formulated to be attached to 10 affinity agents, the particle contains a complete complement of affinity agents if it is attached to 10 affinity agents. A particle containing a complete complement of attached entities may be substantially devoid of non-associated non-dissociable attachment handles. As used herein, the term “incomplete complement,” when used in reference to a particle, refers to the particle not being attached to an intended quantity of entities, such as analytes, affinity agents, detectable labels, docker strands, or tether strands, as set forth herein. For example, if a particle is formulated to be attached to 10 affinity agents, the particle contains an incomplete complement of affinity agents if it is attached to 9 or less affinity agents. A particle containing an incomplete complement of attached entities may comprise one or more non-associated non-dissociable attachment handles.

In some embodiments set forth herein, the term “complexed particle” can refer to a particle or macromolecule that is attached to one or more other entities, such as an analyte, an affinity reagent, an oligonucleotide, a docker strand, a tether strand, a small molecule, a detectable label, a component of a receptor-ligand binding pair (e.g., streptavidin-biotin, neutravidin-biotin, SpyCatcher-SpyTag, SnoopCatcher-SnoopTag, SdyCatcher-SdyTag, etc.), or a combination thereof. In some cases, a complexed particle can comprise a single particle or single macromolecule attached to a single analyte. In some cases, a complexed particle can comprise a single particle or macromolecule attached to a plurality of a type of entity, such as a plurality of analytes, a plurality of affinity agents, a plurality of oligonucleotides, a plurality of tether strands, a plurality of small molecules, a plurality of detectable labels, a plurality of receptor-ligand binding pair components, or a combination thereof.

As used herein, the term “conformational state,” when used in reference to a molecule or particle, refers to the shape or proportionate dimensions of the molecule or particle. At the molecular level conformational state can be characterized by the spatial arrangement of a molecule that results from the rotation of its atoms about their bonds. The conformational state of a macromolecule, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A molecule or particle having a given composition may take on more than one conformational state with or without changes to its composition. For example, a protein having a given amino acid sequence (i.e. protein primary structure) may take on different conformations at the secondary, tertiary or quaternary level, and a nucleic acid having a given nucleotide sequence (i.e., nucleic acid primary structure) may take on different conformations at the secondary, tertiary or quaternary level.

The term “comprising” is intended herein to be open-ended, including not only the recited elements, but further encompassing any additional elements.

In some embodiments set forth herein, the term “directly attached” can refer to a first entity (e.g., a particle or macromolecule) and a second entity (e.g., an analyte, an affinity reagent, an oligonucleotide, a docker strand, a tether strand, a small molecule, a detectable label, a component of a receptor-ligand binding pair) being joined by a binding interaction (e.g., a covalent or non-covalent binding interaction) between the structure of the first entity and the structure of the second entity. Conversely, the term “indirectly attached” can refer to a first entity and a second entity being joined by an intermediary entity that is joined to both the first entity and the second entity. For example, an analyte may be directly attached to a particle by a covalent bond between a functional group attached to the analyte and a functional group attached to the particle. Further, the particle may be directly attached to an oligonucleotide by a covalent bond between a functional group attached to the oligonucleotide and a functional group attached to the particle. Accordingly, the oligonucleotide is indirectly attached to the analyte due to the intermediary particle. An attachment handle that facilitates attachment of a first entity to a second entity may be considered an intermediary entity if it has a molecular weight of 1 kiloDalton (1 kDa) or more. A linking moiety (e.g., a polymer strand, a nanoparticle) that facilitates attachment of an attachment handle to an entity may be considered an intermediary entity if it has a molecular weight of 1 kiloDalton (1 kDa) or more.

As used herein, the term “dissociable,” when used in reference to an attachment handle, refers to the attachment handle being configured to form binding interactions with a complementary attachment handle that can be dissociated (spontaneously or induced) in an average time length that is less than or equal to the time length of a process, method, or step thereof, as set forth herein. For example, an attachment handle and its complementary attachment handle may be considered dissociable if they are expected to dissociate in the presence of a dissociation medium (e.g., a fluid comprising a denaturant or chaotrope) during a 10-minute incubation. A dissociable binding interaction between an attachment handle and its complementary attachment handle may dissociate in no more than about 1 day, 12 hours, 6 hours, 3 hours, 1 hour, 30 minutes, 15 minutes, 10 minutes, 5 minutes, 1 minute, 30 seconds, 15 seconds, 10 seconds, 1 second, or less than 1 second. A dissociable attachment handle may be configured to form a covalent binding interaction or a non-covalent binding interaction. As used herein, the term “non-dissociable,” when used in reference to an attachment handle, refers to the attachment handle being configured to form binding interactions with a complementary attachment handle that can not be dissociated, or can dissociate (spontaneously or induced) in an average time length that is greater than or equal to the time length of a process, method, or step thereof, as set forth herein. For example, an attachment handle and its complementary attachment handle may be considered non-dissociable if they are expected to remain associated in the presence of a dissociation medium (e.g., a fluid comprising a denaturant or chaotrope) during a 10-minute incubation. A non-dissociable binding interaction between an attachment handle and its complementary attachment handle may remain associated for at least about 1 minute, 10 minutes, 15 minutes, 30 minutes, 1 hour, 3 hours, 6 hours, 12 hours, 1 day, or more than 1 day. A non-dissociable attachment handle may be configured to form a covalent binding interaction or a non-covalent binding interaction.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the terms “label” and “detectable label” refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the terms “linker” and “linking moiety” refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

As used herein, the term “nucleic acid nanoparticle” refers to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be more dense than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g. DNA nanoballs), nucleic acid nanotubes (e.g. DNA nanotubes), and nucleic acid origami (e.g. DNA origami). A SNAP may be functionalized to include one or more reactive handles or other moieties. A SNAP may comprise one or more incorporated residues that contain reactive handles or other moieties (e.g., modified nucleotides).

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the terms “protein” and “polypeptide” refer synonymously to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the terms “type” and “species,” when used in reference to a subset of analytes, refer synonymously to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

The embodiments set forth below and recited in the claims can be understood in view of the above definitions.

### Separation of Heterogeneous Particle Mixtures

The present disclosure provides methods and systems for obtaining populations of particles that are enriched for desired forms of the particle. Accordingly, methods of the present disclosure may comprise the steps of: (i) providing a heterogeneous particle mixture, in which the particles of the heterogeneous particle mixture vary amongst each other with respect to size, structure, and/or morphology; and (ii) removing particles having an undesired form from the heterogeneous particle mixture, thereby providing an enriched particle mixture.

Methods and systems for enriching particle mixtures may be especially useful for complexed particles, such as particle formed by the attachment of one or more molecules to a particle. For example, particles enriched by the present method may include: (1) one or more small molecules (e.g., molecules having a molecular weight <1000 Daltons (Da)) attached to a particle or macromolecule; (2) one or more biomolecules attached to a particle or macromolecule; and (3) one or more macromolecules (e.g., molecules having a molecular weight of at least 1000 Da).

Methods of forming complexed particles may inherently produce pluralities of particles that are heterogeneous in some respect. Formation of complexed particles by attachment of two or more entities together may form particles that are complete or properly formed, incomplete or partially-formed particles, over-formed particles (e.g., complexed particles containing additional bound entities exceeding their design), and unreacted complexed particle precursors. Accordingly, a method of the present disclosure may include a step of providing a heterogeneous particle mixture comprising a mixture of desired complexed particles and undesired complexed particles. With respect to a complexed particle design, a desired complexed particle may be any complexed particle that has a size, structure, or morphology that conforms to the intended size, structure, or morphology of the complexed particle design. With respect to the same complexed particle design, an undesired particle may be any particle that has a size, structure, or morphology that deviates from the intended size, structure, or morphology of the complexed particle design.

In some cases, the intended design of a complexed particle can comprise a single entity attached to a single particle. For example, a complex particle may be formed by attaching a single molecule (e.g., a single analyte, a single affinity reagent) to a single particle. Accordingly, a desired complexed particle may be a complexed particle comprising only one particle attached to only one entity. Likewise, an undesired complexed particle may be a particle attached to no molecule, or a complexed particle attached to two or more molecules.

In some cases, the intended design of a complexed particle can be two or more entities attached to a single particle. A complexed particle may have a precise designed configuration (e.g., a single particle attached to exactly two entities; a single particle attached to exactly three entities, etc.). In this example, a desired complexed particle may be a complexed particle comprising only one particle attached to exactly two entities, and an undesired particle may be a complexed particle comprising only one particle attached to 0, 1, 3, or more than 3 entities. A complexed particle may have a designed dispersity (e.g., a single particle attached to 2, 3, or 4 entities). In this example, a desired complexed particle may be a complexed particle comprising only one particle attached to 2, 3, or 4 entities, and an undesired complexed particle may be a complexed particle comprising only one particle attached to 0, 1, 5, or more than 5 entities.

A method of the present disclosure may be provided a complexed particle comprising two or more different entities bound to a particle. For example, the design of a complexed particle may be a particle attached to two identical affinity agents and two identical tether strands. Accordingly, a desired particle may be a complexed particle comprising only one particle attached to 2 affinity agents and attached to 2 tether strands, and an undesired particle may be a single particle attached to 0, 1, 3, or more than 3 affinity agents, and/or 0, 1, 3, or more than 3 tether strands. A complexed particle can comprise a particle attached to only one of a first type of entity and a plurality of a second type of entity, in which the firs type of entity and the second type of entity differ. For example, a complexed particle can comprise a particle attached to only one analyte, two or more docker strands, and two or more pendant single-stranded oligonucleotides.

A method of the present disclosure may include a step of providing a heterogeneous particle mixture, in which the heterogeneous particle mixture comprises a mixture of desired particles and undesired particles. A method of the present disclosure may include a step of forming an enriched particle mixture, in which the enriched particle mixture has an increased fraction of desired particles relative to the heterogeneous particle mixture from which the enriched particle mixture is derived. Degree of enrichment for a particle mixture may be determined with respect to the particles present in a heterogeneous particle mixture, excluding any precursor molecules or other reagents that may be present in the heterogeneous particle mixture. FIG. 7 provides an example of particle enrichment. The left configuration contains a heterogeneous particle mixture. The heterogeneous particle mixture contains 7 desired complexed particles, each desired particle having only one particle 710 attached to only one entity 720. The heterogeneous particle mixture also comprises 7 undesired particles, including 3 particles 710 that are not attached to at least 1 entity, 2 particles 710 attached to 2 entities 720, and 2 particles 710 attached to 3 entities 720. The heterogeneous particle mixture further comprises 4 unattached entities 720. The heterogeneous particle mixture contains 14 total particles, so the heterogeneous particle mixture contains 50% desired particles and 50% undesired particles. The heterogeneous particle mixture undergoes an enrichment method, as set forth herein, thereby providing a retained enriched particle mixture (top right) and a discarded fraction (bottom right). The enriched particle mixture contains the 7 desired complexed particles plus one undesired particle, thereby containing 87.5% desired particles.

A heterogeneous particle mixture may be provided, in which the heterogeneous particle mixture contains at least about 5%, 10%, 15%, 20%, 30%, 40%, 50%, 60%, 70%, 80%, 90%, or more than 90% desired particles. Alternatively or additionally, a heterogeneous particle mixture may be provided, in which the heterogeneous particle mixture contains no more than about 90%, 80%, 70%, 60%, 50%, 40%, 30%, 20%, 15%, 10%, 5%, or less than 5% desired particles.

A heterogeneous particle mixture may comprise an amount of a particular form of an undesired particle. For example, a heterogeneous particle mixture that was designed to contain single particles attached to only one analyte may comprise undesired particles having a particle attached to no analytes or two or more analytes. A heterogeneous particle mixture may be provided with at least about 0.01%, 0.1%, 0.5%, 1%, 2%, 3%, 4%, 5%, 10%, 20%, or more than 20% of a form of an undesired particle. Alternatively or additionally, a heterogeneous particle mixture may be provided with no more than about 20%, 10%, 5%, 4%, 3%, 2%, 1%, 0.5%, 0.1%, 0.01%, or less than 0.01% of a form of an undesired particle.

A method set forth herein may form an enriched particle mixture, in which the enriched particle mixture contains at least about 50%, 60%, 70%, 80%, 90%, 95%, 99%, 99.9%, or more than 99.9% desired particles. Alternatively or additionally, a method set forth herein may form an enriched particle mixture, in which the enriched particle mixture contains no more than about 99.9%, 99%, 95%, 90%, 80%, 70%, 60%, 50%, or less than 50% desired particles.

An enriched particle mixture may be depleted with respect to a particular form of undesired particle. For example, a particle mixture that is to be enriched for single particles attached to only one analyte may be depleted with respect to particles attached to zero analytes and/or two or more analytes. A method set forth herein may form an enriched particle mixture, in which the enriched particle mixture contains no more than about 10%, 5%, 4%, 3%, 2%, 1%, 0.5%, 0.1%, 0.01%, 0.001%, or less than 0.001% of a form of an undesired particle.

Methods of the present disclosure may be particularly well-suited for solid supports that can be provided in a mobile phase or a fluid phase, such as beads, particles, and nanoparticles. Accordingly, a method may include a step of mixing, combining, solvating, and/or suspending a solid support with a fluid phase comprising a heterogeneous particle mixture, as set forth herein. Alternatively, a solid support may be provided as a stationary phase, such as an array or a chromatographic packing. Accordingly, a method may include a step of contacting a fluid phase comprising a heterogeneous particle mixture, as set forth herein, with a stationary phase solid support.

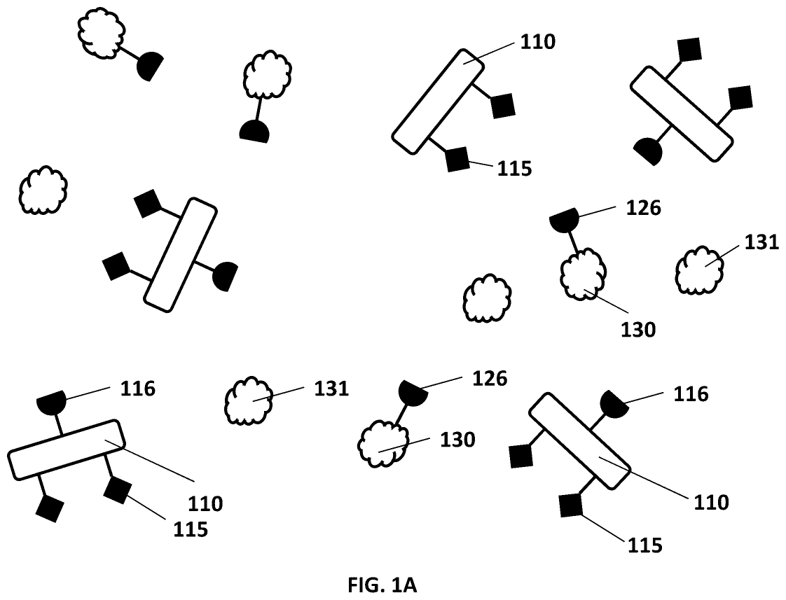

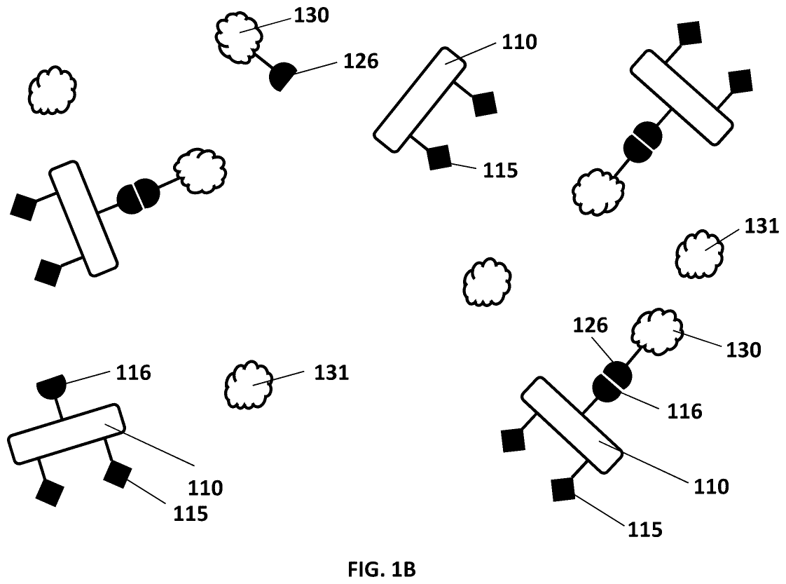

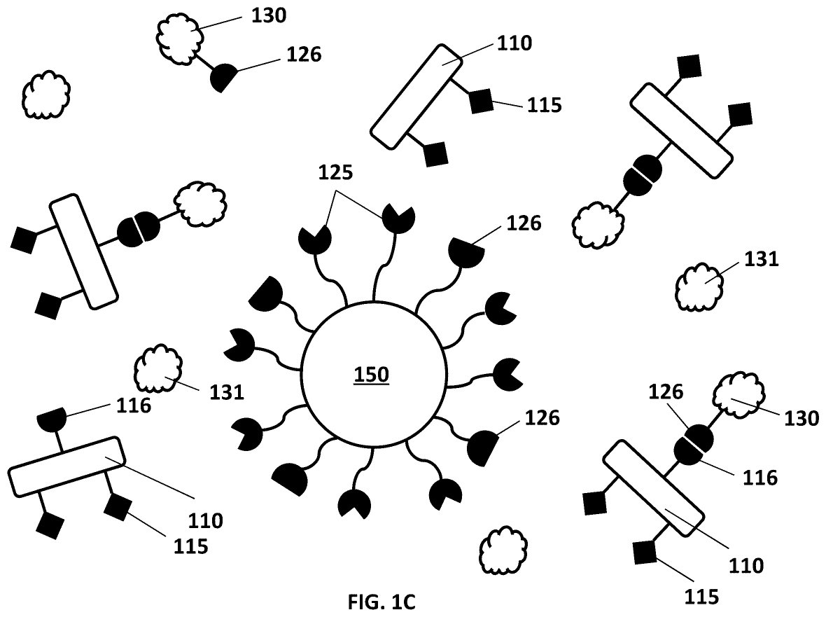

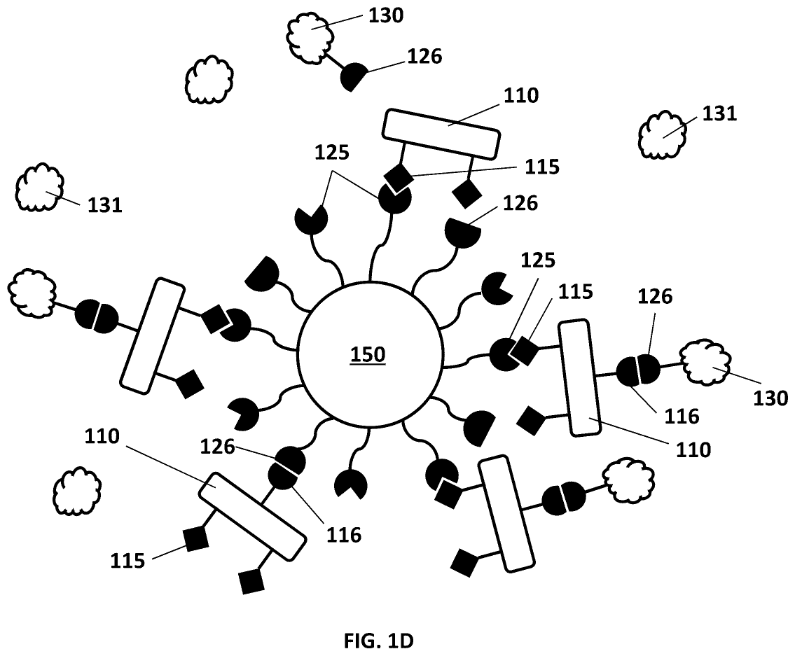

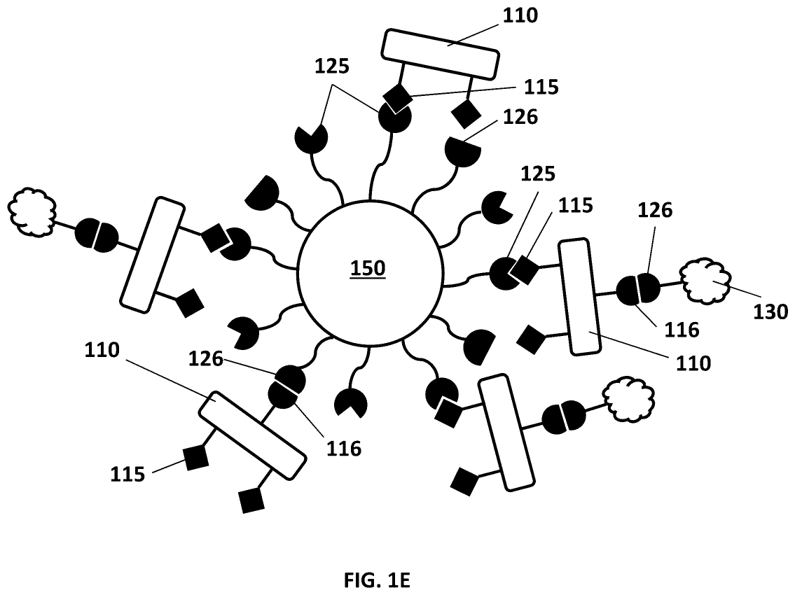

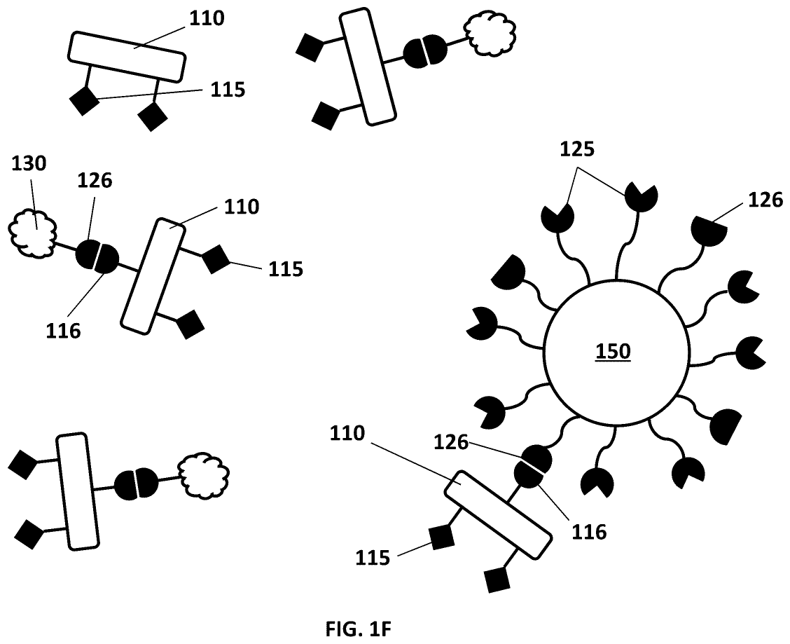

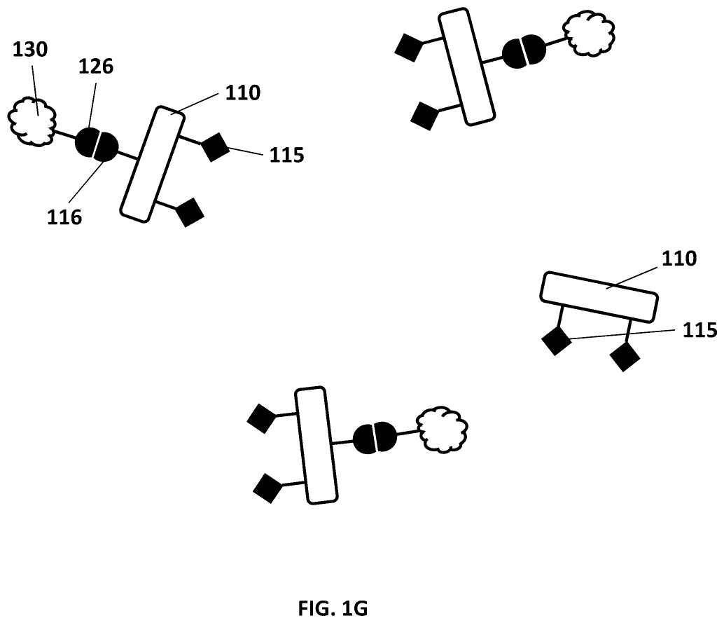

FIGS. 1A-1G illustrate a method for separating partially-formed complexed particles from fully-formed complexed particles in a heterogeneous particle mixture. FIG. 1A depicts a plurality of particles 110 contacted to a plurality of molecules 130 that are attached to complementary non-dissociable attachment handles 126, and a plurality of molecules 131 that are not attached to an attachment handle. Individual particles 110 of the plurality of particles 110 comprise dissociable attachment handles 115 and a non-dissociable attachment handle 116. One macromolecule is not attached to a non-dissociable attachment handle 115 (top center). FIG. 1B depicts the formation of a heterogeneous particle mixture, in which molecules 130 have become attached to particles 110 by coupling of complementary non-dissociable attachment handles 126 attached to the molecules 130 to non-dissociable attachment handles 116 attached to the particles 110, thereby forming complexed particles. The particle 110 that does not comprise a non-dissociable attachment handle 116 is not coupled to a molecule 130. Further, a particle 110 that does comprise a non-dissociable attachment handle 116 is not coupled to a molecule 130 (lower left of FIG. 1).

Continuing with FIG. 1C, a solid support 150 is contacted to the heterogeneous particle mixture. The solid support 150 comprises a plurality of complementary dissociable attachment handles 125 and at least one complementary non-dissociable attachment handles 126. FIG. 1D depicts a configuration formed after the solid support 150 is contacted to the heterogeneous particle mixture. The complexed particles have attached to the solid support 150 by coupling of dissociable attachment handles 115 of the complexed particles to complementary dissociable attachment handles 125 attached to the solid support 150. The particle 110 that does not comprise a non-dissociable attachment handle 116 may attach to the solid support 150 by coupling of dissociable attachment handles 115 attached to the particle 110 to complementary dissociable attachment handles 125 attached to the solid support 150. The particle 110 with an unattached non-dissociable attachment handle 116 can attach to the solid support by coupling of the non-dissociable attachment handle 116 to a complementary non-dissociable attachment handle 126 attached to the solid support 150. FIG. 1E depicts a configuration in which fluid-phase moieties (e.g., unattached molecules 131) have been rinsed from contact with the solid support 150.

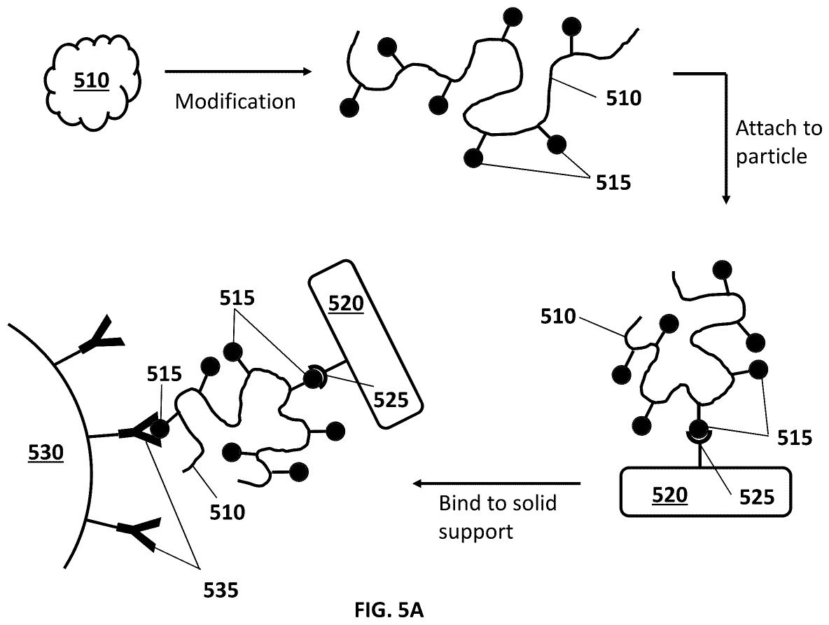

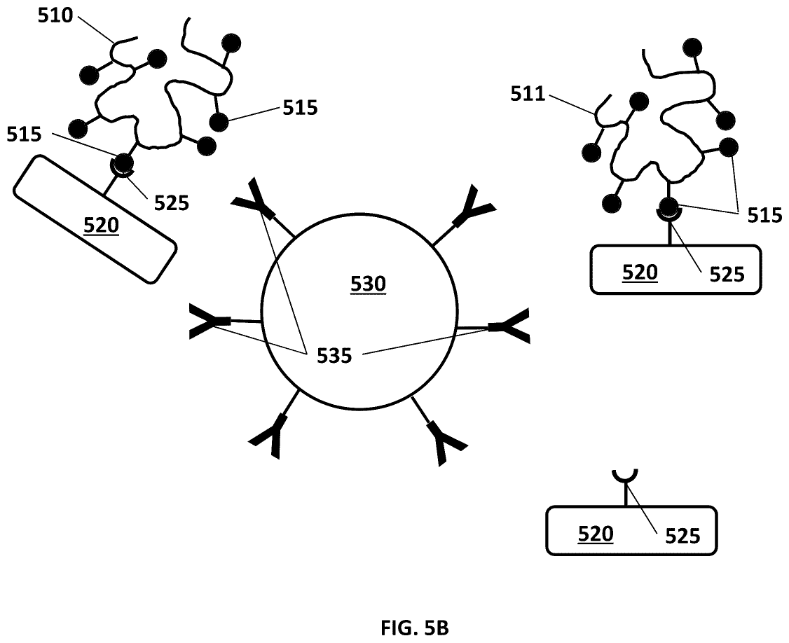

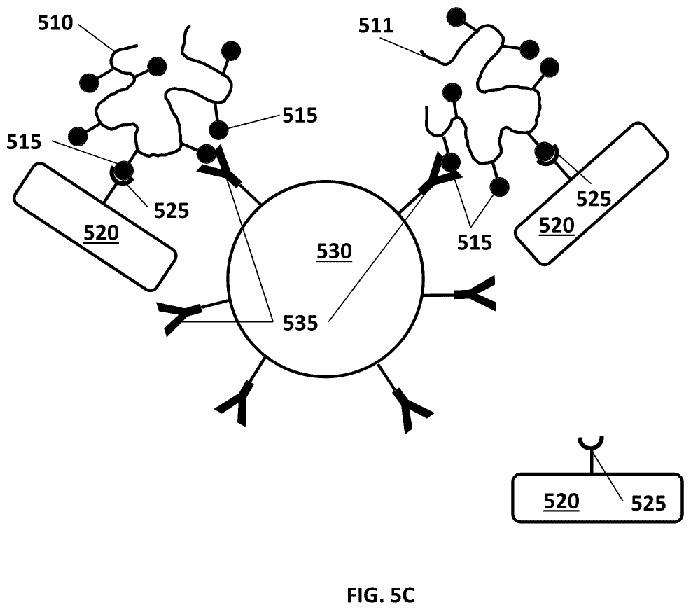

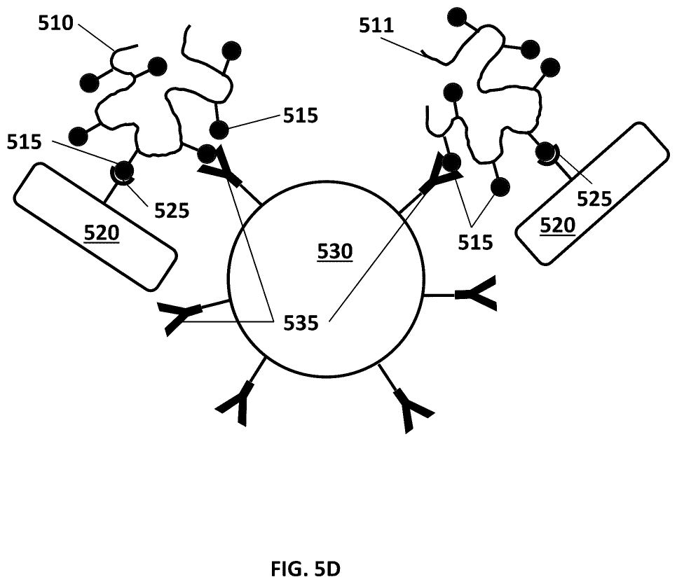

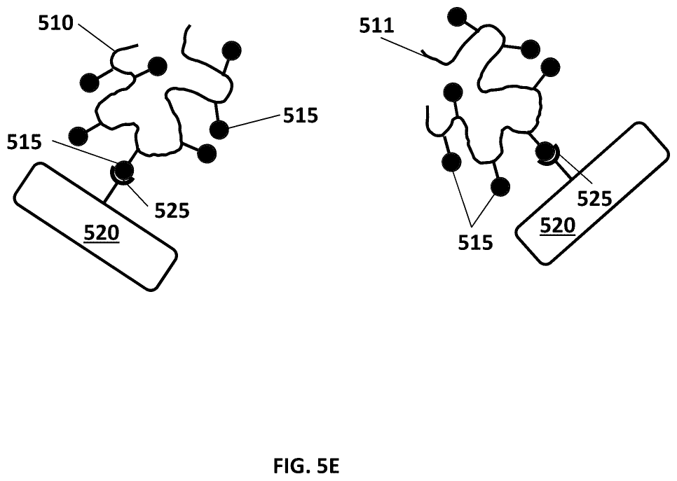

Continuing with FIG. 1F, after rinsing fluid-phase moieties from the solid support 150, particles 110 may be eluted from the solid support 150 by dissociation of the complementary dissociable attachment handles 125 from the dissociable attachment handles 115. FIG. 1G depicts a final step, in which the eluted particles 110 have been removed from contact with the solid support 150. The enriched particle mixture is now substantially devoid of unbound molecules 131 or macromolecules 110 that have not been attached to molecules 130. Optionally, the enriched particle mixture may undergo a subsequent separation process to remove any particles 110 that are not attached to a molecule 130 (for example, the process depicted in FIGS. 5B-5E).

FIG. 5A depicts an alternative method of forming a complexed particle and attaching it to a solid support. An analyte 510 can be modified to attach a plurality of attachment handles 515 to the analyte 510. The plurality of attachment handles 515 can include attachment handles that are configured to form covalent binding interactions (e.g., click-type reactants, SpyTag, SnoopTag, SdyTag, etc.) and/or attachment handles that are configured to form non-covalent binding interactions (e.g., biotin, peptide tags, nucleic acid tags, etc.). In a subsequent step, a complexed particle is formed by attaching an attachment handle 515 of the plurality of attachment handles 515 of the analyte 510 to a complementary attachment handle 525 of a particle 520. In a final step, the complexed particle is bound to a solid support 530. The solid support 530 is attached to a plurality of immobilized affinity reagents 535. The affinity reagents 535 have a binding specificity for attachment handles 515 of the modified analyte 510. Accordingly, the complexed particle is coupled to the solid support 530 by binding of an immobilized affinity reagent 535 to an attachment handle 515 of the complexed particle.

FIG. 5A provides a method that includes modification of an analyte 510 to form a plurality of attachment handles 515 on the analyte 510. Preferably, the attachment handles 515 are antigenic such that an affinity reagent 535 can be obtained that binds to the attachment handle 515. Suitable attachment handles 515 can include certain exogenous small molecule functional groups such as click-type reagents. For biomolecules (e.g., polypeptides), suitable attachment handles can include numerous endogenous modifications, such as methylation, phosphorylation, ubiquitination, SUMOylation, etc. Useful attachment handles can also include purification tags, such as avi-tag. Methods for providing affinity reagents against small-molecule or macromolecular binding targets are well known in the art.

FIG. 5B-5E provide a method of enriching a heterogeneous particle mixture utilizing the attachment method provided in FIG. 5A. FIG. 5B depicts a plurality of complexed particles contacted to a solid support 530 as described in FIG. 5A. The heterogeneous particle mixture further comprises a particle 520 with a complementary attachment handle 525 that is unbound. FIG. 5C depicts a subsequent configuration, in which complexed particles have bound to the solid support 530 by binding of attachment handles 515 of the complexed particles to immobilized affinity reagents 535 of the solid support 530. Due to the absence of attachment handles 515, the unattached particle 520 does not bind to the solid support 530. FIG. 5D depicts a subsequent step, in which the solid support 530 and bound complexed particles are separated by a method set forth herein from the fluid phase containing the unattached particle 520. FIG. 5E depicts a final step, in which the complexed particles are eluted from the solid support 530, thereby providing an enriched particle mixture.

Alternatively to the method of FIGS. 5B-5E, the heterogeneous particle mixture may be enriched by providing a solid support that binds to the complementary attachment handles 525 of the particles 520. Preferably, a solid support 530 is provided with immobilized affinity reagents 535, in which the affinity reagents 535 have a binding specificity for the complementary attachment handles 525 when the complementary attachment handles 525 are not bound to an attachment handle 515. In this method, an enriched particle mixture can be provided by separating from the fluid phase the solid support 530 attached to particles 520 by binding of affinity reagents 535 to complementary attachment handles 525 of the particles.

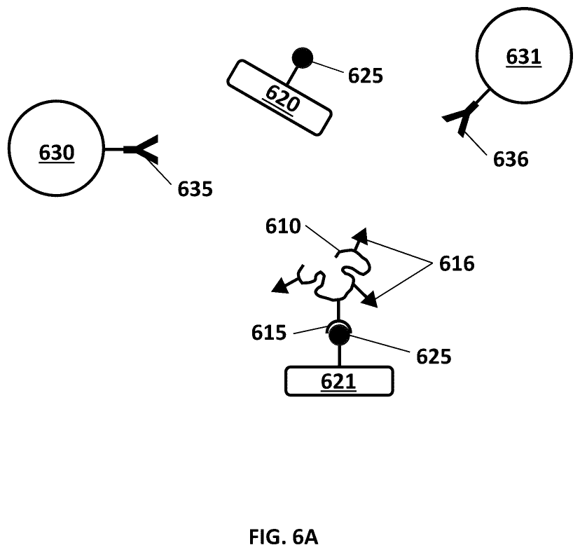

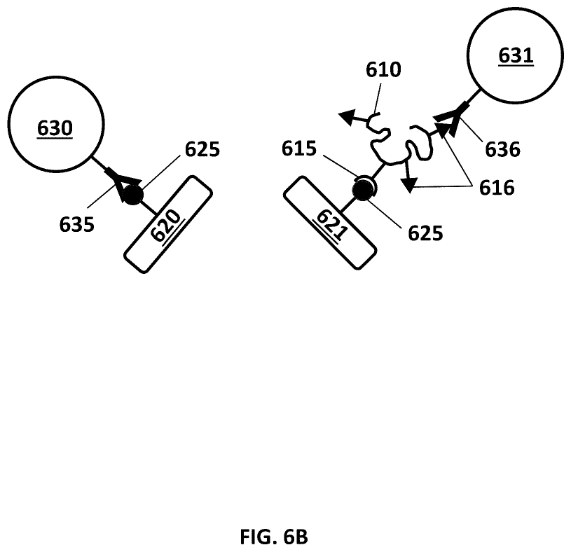

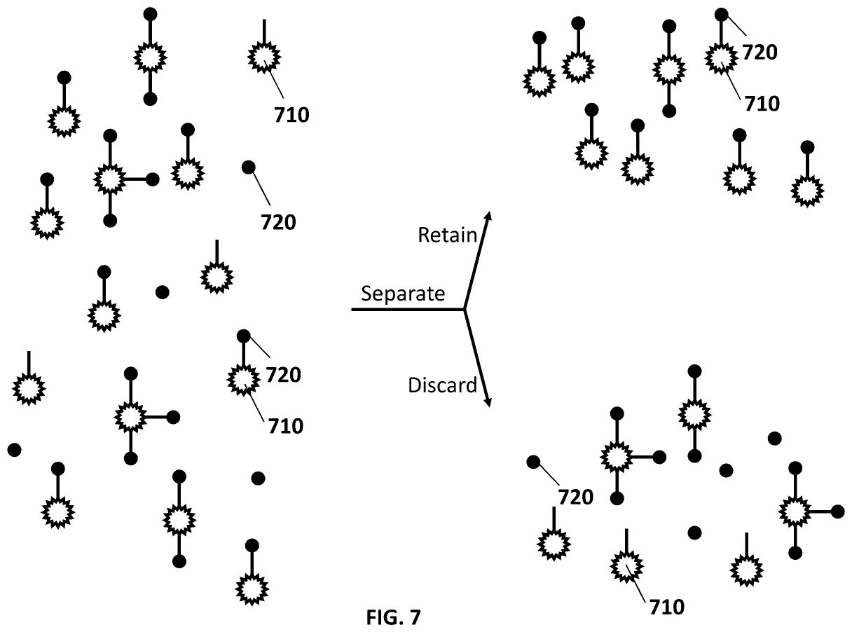

FIGS. 6A-6B illustrate an alternative embodiment of the method of FIGS. 5B-5E, in which two different solid support are utilized to separate desired complexed particles from undesired particles. FIG. 6A depicts a heterogeneous particle mixture that is contacted to two differing solids supports, 630 and 631. The first solid support 630 is attached to one or more affinity reagents 635 that have a binding specificity for unbound first attachment handles 625. The second solid support 631 is attached to one or more affinity reagents 636 that have a binding specificity for unbound second attachment handles 616. The heterogeneous particle mixture comprises a first particle 620 containing a first attachment handle 625. The heterogeneous particle mixture further comprises a second complexed particle, in which the complexed particle comprises a particle 621 attached to an analyte 610 by attachment of a first attachment handle 625 to a complementary first attachment handle 615. The analyte contains a plurality of second attachment handles 616. In some cases, a second attachment handle 616 may be identical to a first complementary attachment handle 615. FIG. 6B depicts a second configuration, in which the first particle 620 is coupled to the first solid support 630 by binding of the affinity reagent 635 to the attachment handle 625. The complexed particle is attached to the second solid support 631 by binding of affinity reagent 636 to the second attachment handle 616. Subsequently, the first solid support 630 can be separated from the second solid support 631, thereby providing an enriched particle mixture containing the complexed particle. For example, the second solid support 631 can be removed from a fluid phase by magnetic separation, electrophoretic separation, settling, or centrifugation, then the first solid support 630 can be removed in the fluid phase, thereby separating the first solid support 630 from the second solid support 631. Although the method depicted in FIGS. 6A-6B utilizes two solid supports to separate a first type of particle from a second type of particle, some methods may only utilize one of the two depicted solid supports.

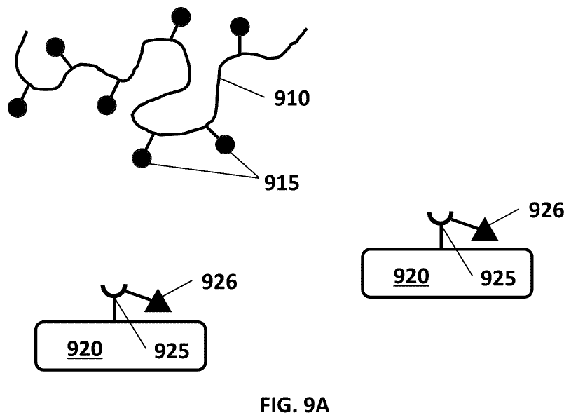

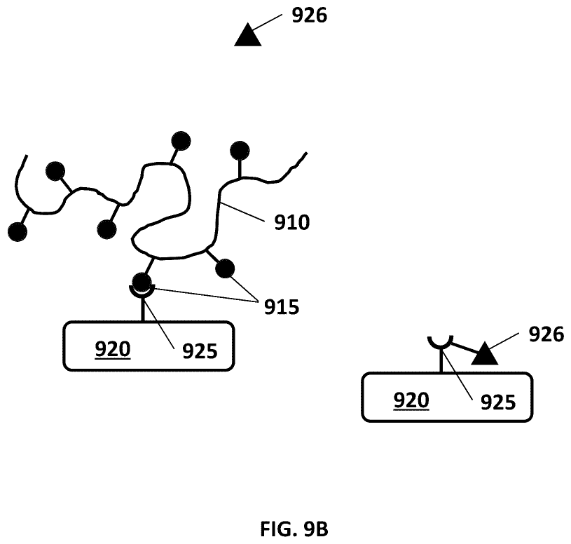

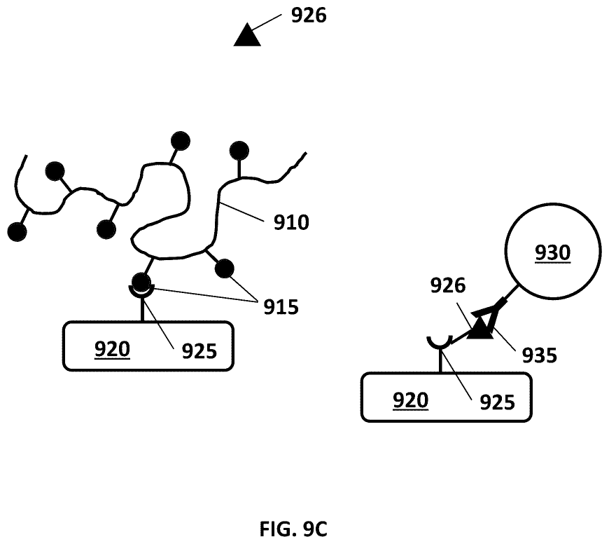

FIGS. 9A-9C depict another method of enriching a heterogeneous particle mixture for desired particles. The method utilizes a releasable handle 926 that is only present on particles 920 that have not attached to an entity (e.g., an analyte, an affinity reagent, a docker strand, a tether strand, etc.). FIG. 9A depicts a configuration of a fluid phase prior to forming a heterogeneous particle mixture by attaching an analyte 910 to a particle 920. The fluid phase contains two particles 920, each particle comprising or attached to a complementary attachment handle 925. In an initial configuration, each complementary attachment handle 925 is attached to a releasable handle 926. The fluid phase also comprises an analyte 910, in which the analyte 910 comprises a plurality of attachment handles 915. FIG. 9B depicts a configuration after forming the heterogeneous particle mixture in the fluid phase. The analyte 910 has coupled to a particle 920 by attachment of an attachment handle 915 of the analyte 910 to the complementary attachment handle 925 of the particle 920. The attachment of the attachment handle 915 to the complementary attachment handle 925 dissociates the releasable handle 926 into the fluid phase. FIG. 9C depicts a subsequent configuration of the fluid phase, in which a solid support 930 is introduced into the fluid phase. An affinity reagent 935 is immobilized on the solid support 930. The affinity reagent 935 has a binding specificity for the releasable handle 926. Accordingly, the particle that is not attached to the analyte 910 is bound by the affinity reagent 935 immobilized on the solid support 930. Subsequently, the solid support 930 coupled to the particle 920 can be separated from the fluid phase, thereby providing an enriched particle mixture comprising the particle 920 attached to the analyte 910.

Reagents that contain a releasable handle, such as those described in FIGS. 9A-9C can include Click-to-Release reagents. Examples of such reagents are provided in Wilkovitsch, et al. “Transforming Aryl-Tetrazines into Bioorthogonal Scissors for Systematic Cleavage of trans-Cyclooctenes.” Ang. Chemie, Int. Ed., (2024), which is herein incorporated by reference in its entirety. The skilled person will recognize that Click-to-Release reagents may be modified with attachment handles that provide sufficient antigenicity to be efficiently scavenged by a complementary affinity reagent.

The skilled person will readily recognize that methods set forth herein for enriching a heterogeneous particle mixture may be readily combined to provide an enriched particle mixture that contains a greater fraction of a desired particle type. In some cases, two or more solid supports may be provided, each solid support configured to bind to a different desired or undesired particle type. In other cases, a solid support may be attached to two or more different attachment handles or affinity reagents, each attachment handle or affinity reagent configured to bind to a different particle type. In other cases, a solid support may be attached to two or more different attachment handles or affinity reagents, each attachment handle or affinity reagent configured to bind to a same particle type.

An enriched particle mixture may be formed from a heterogeneous particle mixture by a single purification method. An enriched particle mixture may be formed from a heterogeneous particle mixture by multiple purification methods that are performed simultaneously. An enriched particle mixture may be formed from a heterogeneous particle mixture by multiple purification methods that are performed sequentially. It will also be recognized that methods may include additional separation methods that facilitate separation of particles, separation of particle precursors, and/or separation of other small molecules from heterogeneous particle mixtures. Such separation methods can include one or more separation techniques such as high-pressure liquid chromatography, affinity chromatography, size-exclusion chromatography, ion chromatography, tangential flow filtration, dialysis, reverse dialysis, and combinations thereof.

In an aspect, provided herein is a method, comprising: (a) contacting a particle mixture to a solid support, wherein the solid support is attached to a complementary dissociable attachment handle and a complementary non-dissociable attachment handle, and wherein the particle mixture comprises: (i) a first particle attached to a dissociable attachment handle and a non-dissociable attachment handle, wherein the non-dissociable attachment handle is attached to a complementary non-dissociable attachment handle of a binding partner, and (ii) a second particle attached to a dissociable attachment handle and a non-dissociable attachment handle, wherein the non-dissociable attachment handle is unattached, (b) binding the dissociable attachment handle of the first particle to the complementary dissociable attachment handle of the solid support, (c) binding the non-dissociable attachment handle of the second particle to the complementary non-dissociable attachment handle of the solid support, and (d) after performing steps (b) and (c), dissociating the first particle from the solid support.

In another aspect, provided herein is a method, comprising: (a) attaching particles of a particle mixture to a solid support, wherein each particle of the particles is attached to the solid support by coupling of a non-dissociable attachment handle of the particle to a complementary non-dissociable attachment handle of the solid support, (b) separating the solid support attached to the particles from the particle mixture, thereby forming an enriched particle mixture, and (c) after separating the solid support from the particle mixture, attaching the enriched particle mixture to a second solid support.

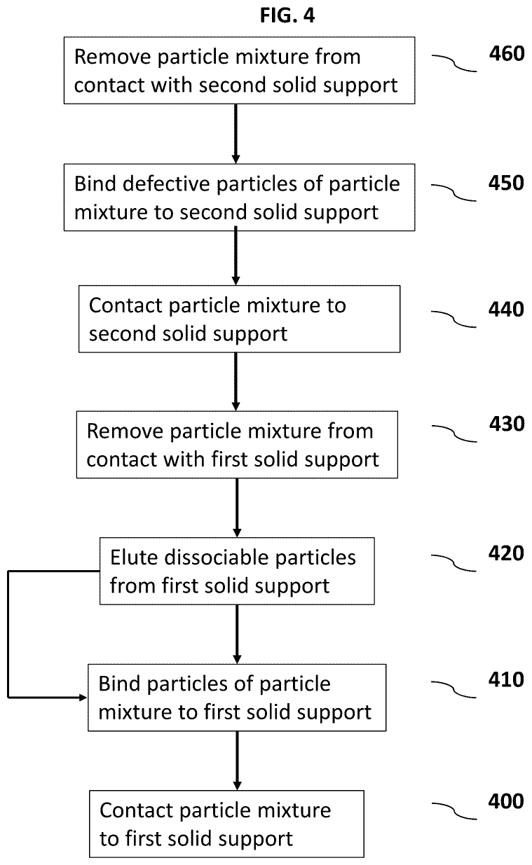

FIG. 4 depicts a schematic of a process for enriching a particle mixture for particles that are attached to a complete complement of entities. In a first step 400, a particle mixture may be contacted to a first solid support, as set forth herein. In a second step 410, particles of the particle mixture, including one or more particles that have a complete complement of attached entities and one or more particles that lack one or more attached entities, may bind to the first solid support. In a third step 420, dissociable particles may be eluted from the first solid support. Optionally, steps 410 and 420 may be repeated one or more times to increase a quantity of particles lacking one or more attached entities that are non-dissociably bound to the first solid support. In a fourth step 430, dissociable particles may be removed from contact with the first solid support, thereby providing an enriched particle mixture containing fewer particles having an incomplete complement of attached entities. Optionally, in a fifth step 440, the enriched particle mixture may be contacted to a second solid support that is configured to bind one or more particles containing a defect, as set forth herein. In an optional sixth step 450, particles of the particle mixture may bind to the second solid support. In an optional seventh step 460, particles may be removed from contact with the second solid support, thereby providing an enriched particle mixture containing fewer defective particles in the enriched particle mixture. Alternatively, steps 440 to 460 may be performed before steps 400 to 430.

A method may comprise a step of forming a particle mixture. In some cases, forming a particle mixture may comprise attaching entities to particles, thereby forming a particle mixture comprising at least one particle attached to an entity and at least one particle that is not attached to an entity. In particular cases, forming a particle mixture may comprise attaching entities to particles, thereby forming a particle mixture comprising at least one particle attached to one and only one entity, and at least one particle that is not attached to an entity. In other cases, forming a particle mixture may comprise attaching entities to particles, thereby forming a particle mixture comprising at least one particle attached to a plurality of entities and at least one particle that is not attached to a plurality of entities. In particular cases, forming a particle mixture may comprise attaching entities to particles, thereby forming a particle mixture comprising at least one particle attached to an intended quantity of entities and at least one particle that is not attached to the intended quantity of entities.

Forming a particle mixture may comprise the steps of: (i) contacting an entity comprising a complementary non-dissociable attachment handle to a first particle comprising a non-dissociable attachment handle and a second particle comprising the same type of non-dissociable attachment handle as the first particle, and (ii) binding the complementary non-dissociable attachment handle of the entity to the non-dissociable attachment handle of the first particle. In some cases, forming a particle mixture may comprise the steps of: (i) contacting entities each individually comprising a complementary non-dissociable attachment handle to a plurality of particles each individually comprising a non-dissociable attachment handle, and (ii) binding complementary non-dissociable attachment handles of entities to non-dissociable attachment handles of particles of the plurality of particles. A particle mixture may comprise a first plurality of particles comprising a complete complement of attached entities and a second plurality of second particles lacking at least one intended entity (e.g., an absence of at least one analyte, an absence of at least one affinity agent, an absence of at least one detectable label, etc.).

For particle mixtures comprising a first plurality of particles comprising a complete complement of attached entities and a second plurality of second particles lacking at least one intended entity, a method may comprise a step binding the dissociable attachment handles of particles of the first plurality of particles to complementary dissociable attachment handles of a solid support. A method may further comprise a step of binding the non-dissociable attachment handles of particles of the second plurality of particles to complementary non-dissociable attachment handles of the solid support. A method may further comprise a step of dissociating the first particles from the solid support.

In some cases, a particle mixture may comprise a plurality of particles or macromolecules attached to a plurality of entities (e.g., analytes, affinity agents, detectable labels, other macromolecules, etc.). In particular cases, a particle mixture may comprise a plurality of particles, in which individual particles of the plurality of particles are attached to only one entity (e.g., only one analyte, only one affinity agent, only one detectable label, only one other macromolecule, etc.). A method set forth herein may provide a particle mixture that is enriched for particles attached to one and only one entity. After performing a method set forth herein, a particle mixture may comprise a plurality of particles, in which at least about 50%, 55%, 60%, 65%, 70%, 75%, 80%, 85%, 90%, 95%, 99%, 99.9%, 99.99%, 99.999%, or more than 99.999% of particles of the plurality of particles are attached to only one entity. Alternatively or additionally, a particle mixture may comprise a plurality of particles, in which no more than about 99.999%, 99.99%, 99.9%, 99%, 95%, 90%, 85%, 80%, 75%, 70%, 65%, 60%, 55%, 50%, or less than 50% of particles of the plurality of particles are attached to only one entity.

In other cases, a particle mixture may comprise a plurality of particles or macromolecules attached to a plurality of entities (e.g., analytes, affinity agents, detectable labels, other macromolecules, etc.). In particular cases, a particle mixture may comprise a plurality of particles, in which an individual particle of the plurality of particles is attached to a plurality of entities (e.g., a plurality of analytes, a plurality of affinity agents, a plurality of detectable labels, a plurality of other macromolecules, or combinations thereof). A method set forth herein may provide a particle mixture that is enriched for particles having an intended quantity of entities (e.g., a particle with 3 affinity agent attachment handles is intended to be attached to 3 affinity agents). After performing a method set forth herein, a particle mixture may comprise a plurality of particles, in which at least about 50%, 55%, 60%, 65%, 70%, 75%, 80%, 85%, 90%, 95%, 99%, 99.9%, 99.99%, 99.999%, or more than 99.999% of particles of the plurality of particles are attached to an intended quantity of entities. Alternatively or additionally, a particle mixture may comprise a plurality of particles, in which no more than about 99.999%, 990.99%, 99.9%, 99%, 95%, 90%, 85%, 80%, 75%, 70%, 65%, 60%, 55%, 50%, or less than 50% of particles of the plurality of particles are attached to an intended quantity of entities.

A particle of a plurality of particles may comprise a dissociable attachment handle and a non-dissociable attachment handle. A method may comprise contacting the particle having the dissociable attachment handle and the non-dissociable attachment handle to a solid support comprising a complementary dissociable attachment handle and a complementary non-dissociable attachment handle. In some cases, a non-dissociable attachment handle of a particle can comprise a first reactive moiety, in which a complementary non-dissociable attachment handle of a solid support can comprise a second reactive moiety. Accordingly, a method may comprise binding the non-dissociable attachment handle of the particle to the complementary non-dissociable attachment handle of the solid support, in which the binding comprises covalently attaching the first reactive moiety of the particle to the second reactive moiety of the solid support. In particular cases, the first reactive moiety and the second reactive moiety comprise click-type reagents. In other particular cases, the first reactive moiety and the second reactive moiety can comprise a receptor-ligand binding pair that is configured to form an isopeptide bond (e.g., SpyCatcher-SpyTag, SdyCatcher-SdyTag, and SnoopCatcher-SnoopTag, etc.).

In other cases, a non-dissociable attachment handle of a particle may comprise a first non-reactive moiety, in which the complementary non-dissociable attachment handle of a solid support can comprise a second non-reactive moiety. Accordingly, binding the non-dissociable attachment handle of the particle to the complementary non-dissociable attachment handle of the solid support can comprise non-covalently attaching the first non-reactive moiety to the second non-reactive moiety. In particular cases, the first non-reactive moiety and the second non-reactive moiety can comprise a receptor-ligand binding pair (e.g., streptavidin-biotin, avidin-biotin, etc.).

A particle of a plurality of particles may comprise a dissociable attachment handle and a non-dissociable attachment handle. A method may comprise contacting the particle having the dissociable attachment handle and the non-dissociable attachment handle to a solid support comprising a complementary dissociable attachment handle and a complementary non-dissociable attachment handle. In some cases, a dissociable attachment handle of a particle can comprise a first single-stranded nucleic acid, in which the complementary dissociable attachment handle of a solid support can comprise a second single-stranded nucleic acid. Accordingly, binding the dissociable attachment handle of the particle to the complementary dissociable attachment handle of the solid support can comprise hybridizing the first single-stranded nucleic acid to the second single-stranded nucleic acid (i.e., thereby forming a double-stranded nucleic acid). In such configurations, the first single-stranded nucleic acid of the particle can comprise a first nucleotide sequence that is complementary to a second nucleotide sequence of the second single-stranded nucleic acid. Accordingly, dissociating the particle from the solid support can comprise de-hybridizing the first nucleic acid strand from the second nucleic acid strand (i.e., de-hybridizing the double-stranded nucleic acid).

Systems of dissociable attachment handles and complementary dissociable attachment handles need not be limited to nucleic acids. Other useful interactions can include protein-antibody, protein-aptamer, and protein-nucleic acid. In some cases, a dissociable interaction can be formed between a first reactive moiety and a second reactive moiety, in which the first reactive moiety and the second reactive moiety are configured to form a photocleavable covalent bond. Accordingly, dissociating the particle from the solid support can comprise dissociating the photocleavable covalent bond with a photon of light.

Methods of the present disclosure can facilitate separation of particles that have bound to a complete complement of entities from particles that have not bound to a complete complement of entities. A method may comprise a step of, after dissociating a particle from a solid support (e.g., via dissociation of a binding interaction between a complementary dissociable attachment handle of the solid support and a dissociable attachment handle of the particle) removing or separating a particle or particles from contact with the solid support. In some cases, removing or separating a particle or particles from contact with a solid support may comprise removing a fluid containing the particle or particles from contact with the solid support (e.g., via decanting or pipetting the fluid).

In some cases, removing the fluid containing the particle or particles from contact with the solid support can comprise separating the one or more discrete units (e.g., beads, particles) from the fluid containing the particle or particles. The one or more discrete units may comprise one or more magnetic beads or electrically-charged beads or particles. Accordingly, separating the one or more discrete units from the fluid containing the particle or particles can comprise separating the one or more discrete units from the fluid containing the particle or particles by magnetophoresis or electrophoresis. Alternatively, the one or more discrete units can have a density greater than a density of the fluid containing the particle or particles. Accordingly, separating the one or more discrete units from the fluid containing the particle or particles can comprise sedimenting or centrifuging the one or more discrete units from the fluid containing the particle or particles. A method may comprise the steps of: i) transferring one or more discrete units of a solid support to the surface of a second solid support, and ii) removing a fluid containing a particle or particles from contact with the second solid support and the one or more discrete units.

A method may further comprise a step of exchanging the fluid containing a particle or particles with a second fluid. A second fluid may be configured to stabilize or preserve the particle or particles for transfer, storage, or utilization in another process. In some cases, a second fluid may comprise an excipient agent, such as a cryoprotectant, a biocidal agent, a chaotropic agent, a denaturing agent, a reactive species inhibitor, an anti-aggregant, an enzymatic inhibitor, a molecular stability promoter, or a combination thereof.

Alternatively, a method may further comprise separating a particle or particles from a fluid after the fluid has been removed from contact with the solid support. For example, separating the particle or particles from the fluid can comprise lyophilizing or crystallizing the particle or particles.

Various types of solid supports may be useful for methods of the present disclosure. In some cases, it may be useful to provide a solid support that can be provided in a mobile phase or a fluid phase (e.g., a solid support that can be provided as a suspension of solution). Exemplary mobile or fluid-phase solid supports can include beads, microbeads, nanobeads, microparticles, nanoparticles, or combinations thereof. Accordingly, a solid support may be mixed together with a plurality of particles to form a fluid-phase mixture. Alternatively, a solid support can be provided as an immobile phase, such as a solid support or packing material for a chromatography column, tubing, plates, or any other conceivable contiguous solid medium. Accordingly, a plurality of particles may be contacted to an immobile solid support.

A solid support may be provided with a plurality of complementary dissociable attachment handles and a plurality of complementary non-dissociable attachment handles. Methods of immobilizing moieties on solid supports are known in the art and are not particularly limited for the present disclosure. For a solid support that is divided into a plurality of discrete units (e.g., a plurality of beads or particles), each discrete unit of the plurality of discrete units may comprise at least one complementary dissociable attachment handle and/or at least one complementary non-dissociable attachment handle. Alternatively, a first discrete unit of a solid support may comprise only complementary dissociable attachment handles and a second discrete unit of a solid may comprise only complementary non-dissociable attachment handles. A solid support may be provided with a plurality of complementary dissociable attachment handles and a plurality of complementary non-dissociable attachment handles with a ratio of complementary dissociable attachment handles to complementary non-dissociable attachment handles of at least about 0.000001, 0.00001, 0.0001, 0.001, 0.01, 0.1, 0.5, 1, 1.5, 2, 5, 10, 50, 100, 1000, 10000, 100000, 1000000, or more than 1000000. Alternatively or additionally, a solid support may be provided with a plurality of complementary dissociable attachment handles and a plurality of complementary non-dissociable attachment handles with a ratio of complementary dissociable attachment handles to complementary non-dissociable attachment handles of no more than about 1000000, 100000, 10000, 1000, 100, 50, 10, 5, 2, 1.5, 1, 0.5, 0.1, 0.01, 0.001, 0.0001, 0.00001, 0.000001, or less than 0.000001.

A solid support may be provided with a plurality of complementary dissociable attachment handles and/or a plurality of complementary non-dissociable attachment handles that are in molar excess relative to a quantity of particles of a plurality of particles (e.g., relative to a quantity of particles having a complete complement of attached entities, relative to a quantity of particles having an incomplete complement of attached entities). A solid support may be provided with a plurality of complementary dissociable attachment handles and/or a plurality of complementary non-dissociable attachment handles that are in molar excess relative to a quantity of particles by a factor of at least about 1.1-fold, 1.5-fold, 2-fold, 5-fold, 10-fold, 20-fold, 50-fold, 100-fold, 500-fold, 1000-fold, or more than 1000-fold excess. Alternatively or additionally, a solid support may be provided with a plurality of complementary dissociable attachment handles and/or a plurality of complementary non-dissociable attachment handles that are in molar excess relative to a quantity of particles by a factor of no more than about 1000-fold, 500-fold, 200-fold, 100-fold, 50-fold, 20-fold, 10-fold, 5-fold, 2-fold, 1.5-fold, 1.1-fold, or less than 1.1-fold excess. It may be advantageous to provide complementary dissociable or non-dissociable attachment handles in a molar excess to ensure highly efficient capture of particles comprising less than a full complement of attached entities.

Alternatively, a solid support may be provided with a plurality of complementary dissociable attachment handles and/or a plurality of complementary non-dissociable attachment handles that are in molar deficit relative to a quantity of particles of a plurality of particles (e.g., relative to a quantity of particles having a complete complement of attached entities, relative to a quantity of particles having an incomplete complement of attached entities). A solid support may be provided with a plurality of complementary dissociable attachment handles and/or a plurality of complementary non-dissociable attachment handles that are in molar deficit relative to a quantity of particles by a factor of at least about 1.1-fold, 1.5-fold, 2-fold, 5-fold, 10-fold, 20-fold, 50-fold, 100-fold, 500-fold, 1000-fold, or more than 1000-fold deficit. Alternatively or additionally, a solid support may be provided with a plurality of complementary dissociable attachment handles and/or a plurality of complementary non-dissociable attachment handles that are in molar deficit relative to a quantity of particles by a factor of no more than about 1000-fold, 500-fold, 200-fold, 100-fold, 50-fold, 20-fold, 10-fold, 5-fold, 2-fold, 1.5-fold, 1.1-fold, or less than 1.1-fold deficit. Providing a molar deficit of complementary dissociable attachment handles or complementary non-dissociable attachment handles may facilitate more rapid uptake of particles on the solid support due to a more rapid drive to equilibrium.

A method may comprise a step of dissociating a particle or particles from a solid support, in which dissociation comprises dissociating a complementary dissociable attachment handle of a solid support from a dissociable attachment handle of a particle. Depending upon the type of dissociable attachment handle utilized, methods of dissociation are known in the art. For example, dissociating a particle or particles from a solid support can comprise contacting the solid support with a chaotropic agent or a denaturing agent (e.g., for dissociating nucleic acid-nucleic acid interactions, protein nucleic acid interactions, or protein-protein interactions). In another example, dissociating a particle or particles from the solid support can comprise heating the solid support. In yet another example, dissociating a particle or particles from the solid support can comprise contacting the solid support with a displacement agent, such as a toehold strand-mediated displacement oligonucleotide. In some cases, a displacement agent can comprise a competitor species. A competitor species may be configured to bind to a dissociable attachment handle of a solid support, thereby displacing an associated particle. For example, a displacement agent can comprise a single-stranded nucleic acid, in which the single-stranded nucleic acid is complementary to a complementary dissociable attachment oligonucleotide of a solid support. In another example, a displacement agent can comprise a peptide, in which the peptide is complementary to a complementary dissociable attachment handle (e.g., an antibody or aptamer) of a solid support. In some cases, a displacement agent can comprise a scavenger species. A scavenger species may be configured to bind to a dissociable attachment handle of a particle, thereby displacing an associated particle from a solid support. For example, the displacement agent can comprise a single-stranded nucleic acid, in which the single-stranded nucleic acid is complementary to the dissociable attachment handle of a particle. In another example, a displacement agent can comprise a peptide, in which the peptide is complementary to a dissociable attachment handle (e.g., an antibody or aptamer) of a particle.

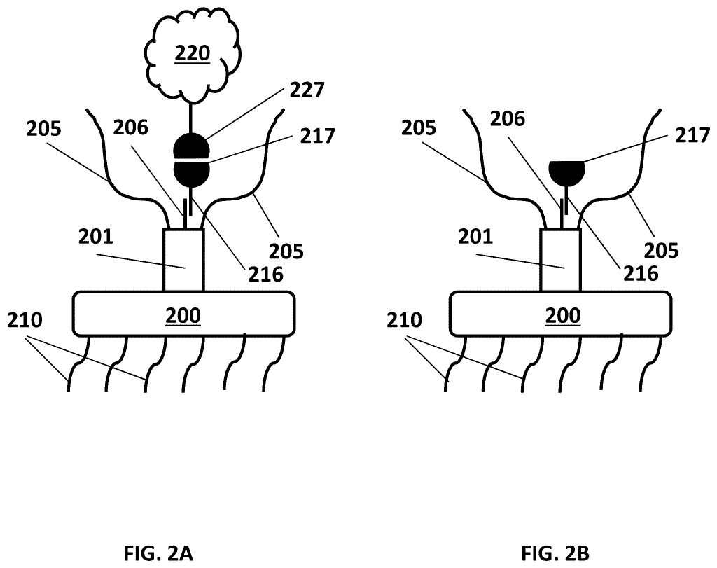

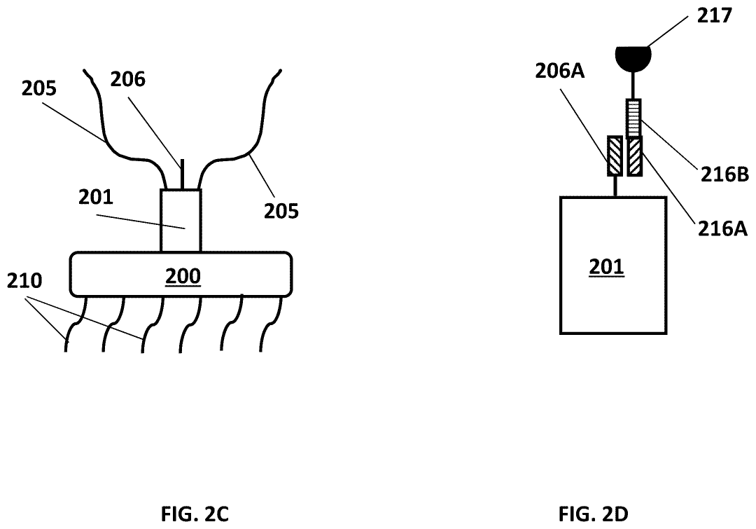

FIGS. 2A-2D illustrate examples of complexed particles comprising analytes that may be useful for some methods set forth herein, such as methods of forming arrays of analytes. FIG. 2A illustrates a particle 200 (e.g., a nucleic acid nanoparticle, a particle substantially devoid of nucleic acids, such as a synthetic polymer particle, a dendrimer, or a branched polymer). The particle 200 optionally comprises a separating group 201 that may increase a separation distance between an analyte 220 and a surface to which the particle 200 is attached. The particle 200 further comprises surface-coupling moieties (e.g., oligonucleotides, receptor-ligand binding pair components, reactive functional moieties) that can facilitate attachment of the particle 200 to a surface. Optionally, the particle 200 comprises one or more docker moieties 205, as set forth herein, optionally attached to the separating groups 201 or the particle 200. The analyte 220 is attached to the particle by a binding interaction between a complementary non-dissociable attachment handle 227 attached to the analyte 220 and a non-dissociable attachment handle 217. The non-dissociable attachment handle 217 is attached to an oligonucleotide 216 that hybridizes to a complementary oligonucleotide 206 of the particle 200, thereby attached the non-dissociable attachment handle 217 to the particle 200.

FIG. 2B depicts a particle 200 that is not attached to an analyte 220. Accordingly, the particle is attached to a non-dissociable attachment handle 217 that is not attached to a complementary non-dissociable attachment handle 227 of an analyte 220. A solid support comprising a complementary non-dissociable attachment handle 227 may be useful for removing the particle of FIG. 2B from a particle mixture. FIG. 2C depicts a particle that does not comprise a non-dissociable attachment handle 217 due to a lack of an oligonucleotide 216 bound to the oligonucleotide 206. Accordingly, the particle 200 may not be captured on a solid support comprising a complementary non-dissociable attachment handle 227. However, the oligonucleotide 206 may be useful for capture by a defect capture handle, as set forth herein.

FIG. 2D depicts a detailed diagram of the separating group 201 of FIGS. 2A and 2B. The separating group 201 may be attached to an oligonucleotide comprising a sequence 206A. The sequence 206A hybridizes to a sequence 216A of an oligonucleotide that is attached to a non-dissociable attachment handle 217. The oligonucleotide containing the sequence 216A further comprises a single-stranded sequence 216B that may be configured to be captured by a dissociable attachment handle of a solid support.

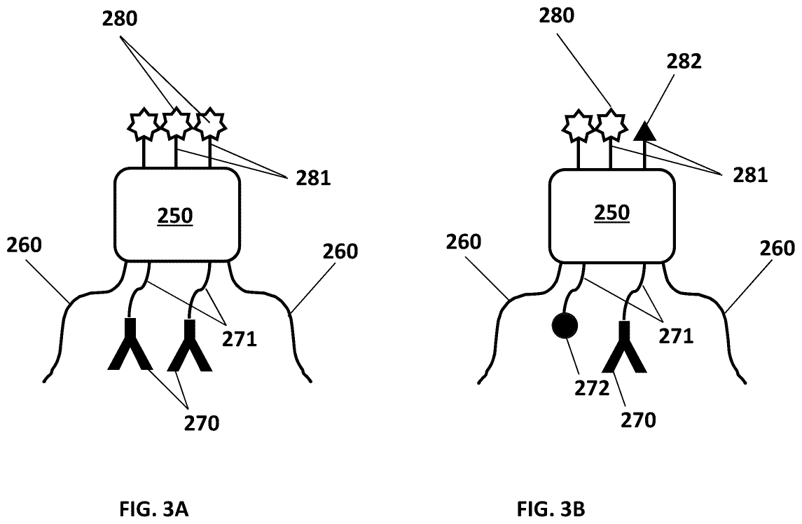

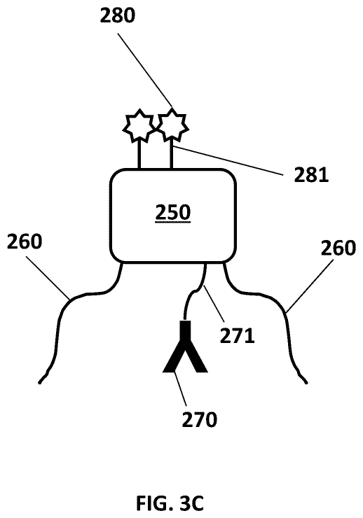

FIGS. 3A-3C illustrate examples of complexed particles comprising affinity agents and/or detectable labels that may be useful for some methods set forth herein, such as methods of detecting analytes of arrays of analytes. FIG. 3A illustrates a particle 250 that is attached to a plurality of affinity agents 270 and a plurality of detectable labels 280. Each affinity agent 270 is attached to the particle 250 by a linking moiety 271. Each detectable label 280 is attached to the particle 250 by a linking moiety 281. The particle 250 is optionally further attached to one or more tether strands 260, as set forth herein.

FIG. 3B depicts a configuration of the particle of FIG. 3A, in which a detectable label 280 is not attached to a linking moiety 281, thereby leaving an unattached non-dissociable attachment handle 282. Further, an affinity agent 270 is not attached to a linking moiety 271, thereby leaving an unattached non-dissociable attachment handle 272. The particle of FIG. 3B may be removed from a particle mixture by contact with a solid support comprising a complementary non-dissociable attachment handle for either the non-dissociable attachment handle 282 or the non-dissociable attachment handle 272, or optionally both complementary non-dissociable attachment handles for both. FIG. 3C illustrates a particle 250 that is further missing the linking moiety 281 and the linking moiety 271. Accordingly, the particle 250 may not be captured on a solid support comprising a complementary non-dissociable attachment handle. However, the region of the particle lacking a linking moiety 281 or a linking moiety 271 may be useful for capture by a defect capture handle, as set forth herein.

A particle mixture may comprise an unattached entity or a plurality thereof. For example, a particle mixture may comprise one or more analytes, affinity agents, docker strands, tether strands, and/or detectable labels that did not attach to a particle, either due to lack of a complementary attachment handle, lack of a particle available for attachment, or failure to attach to a particle during an incubation period. A method may further comprise, before dissociating a particle or particles from a solid support, separating an unattached entity of the one or more unattached entities from the solid support. Separating an unattached entity from a solid support may comprise rinsing or decanting a fluid containing the unattached entity from the solid support. Alternatively, separating an unattached entity from a solid support may comprise separating or extracting the solid support from a fluidic medium containing the unattached entity.

A particle mixture may comprise a non-associable particle, or a plurality thereof. For example, a particle may not comprise a non-dissociable attachment handle that is configured to bind to a complementary attachment handle of an entity. In some cases, a method may comprise a step of, before dissociating a particle or particles from a solid support, separating a non-associable particle of the one or more non-associable particles from the solid support. Separating a non-associable particle from a solid support may comprise rinsing or decanting a fluid containing the non-associable particle from the solid support. Alternatively, separating a non-associable particle from a solid support may comprise separating or extracting the solid support from a fluidic medium containing the non-associable particle.

Alternatively, a non-associable particle may be removed from a particle mixture by contacting the particle mixture with a solid support. In some cases, the solid support can comprise a defect capture handle, in which the defect capture handle is configured to bind to a non-associable particle or a defect thereof. For example, a particle comprising a nucleic acid nanoparticle may be non-associable due to a failure to incorporate an oligonucleotide containing a dissociable attachment handle and/or a non-dissociable attachment handle. In such cases, a defect capture handle could comprise an oligonucleotide comprising a same nucleotide sequence as a nucleotide that was not incorporated into the non-associable particle. In another example, a particle could comprise a defect having a specific morphology, moiety, or epitope due to the failed incorporation of a dissociable attachment handle and/or a non-dissociable attachment handle. In such cases, a defect capture handle could comprise an affinity agent that is configured to bind to the defect of the non-associable particle.

A non-associable particle may be removed from a particle mixture by contacting the particle mixture with a solid support, in which the solid support comprises a dissociable attachment handle and/or a non-dissociable attachment handle or pluralities thereof, and further comprises a defect capture handle or a plurality thereof. Accordingly, a method, as set forth herein, may further comprise a step of, before dissociating a particle comprising a complete complement of attached entities, binding a non-associable particle to a defect capture handle of the solid support.

Alternatively, a non-associable particle may be removed from a particle mixture by contacting the particle mixture with a second solid support, in which the second solid support comprises a defect capture handle or a plurality thereof. In some cases, the second solid support can comprise a plurality of discrete units (e.g., beads, particles, etc.). The plurality of discrete units of the second solid support may be mixed with a plurality of discrete units of a first solid support comprising a dissociable attachment handle and/or a non-dissociable attachment handle or pluralities thereof. Accordingly, a method may comprise binding a non-associable particle to a defect capture handle of a discrete unit of the second solid support and binding one or more particles to a dissociable attachment handle and/or a non-dissociable attachment handle of a discrete unit of the first solid support. Alternatively, a particle mixture comprising a non-associable particle may be contacted to the second solid support before or after contacting the particle mixture to the first solid support. A method may further comprise a step of removing and/or separating a particle mixture from contact with the second solid support. Methods for removing and/or separating particle mixtures from solid supports are described elsewhere herein.

A method may comprise a step of incubating a particle mixture, as set forth herein, with a solid support, thereby associating particles of the particle mixture to the solid support. A particle mixture may be incubated with a solid support for at least about 1 second (s), 30 s, 1 minute (min), 5 mins, 10 mins, 15 mins, 30 mins, 1 hour (hr), 6 hrs, 12 hrs, 24 hrs, or more than 24 hrs. Alternatively or additionally, a particle mixture may be incubated with a solid support for no more than about 24 hrs, 12 hrs, 6 hrs, 1 hr, 30 mins, 15 mins, 10 mins, 5 mins, 1 min, 30 s, 1 s, or less than 1 s. If the solid support comprises a plurality of discrete units, incubating the particle mixture with the solid support can further comprise mixing the particle mixture with the discrete units of the solid support (e.g., mixing in a fluid phase). Mixing of a solid support with a plurality of particles can include non-quiescent fluid flow, such as flow of particles across the solid support, or flow of the solid support through a fluid containing particle. Alternatively, a fluid may be quiescently incubated with a solid support.

It will be recognized that a particle in a particle mixture having an incomplete complement of attached entities may comprise a dissociable attachment handle and a non-dissociable attachment handle. Accordingly, the particle may bind to a solid support in a preferred configuration (via a non-dissociable binding interaction) or an unwanted configuration (via a dissociable binding interaction). It may be preferable to dissociate such particles from a solid support one or more times, thereby providing additional opportunities for particles having an incomplete complement of attached entities to be attached to a solid support by a non-dissociable interaction. A method may further comprise one or more steps of: (i) dissociating one or more particles comprising unattached non-dissociable attachment handles from a solid support, and (ii) after dissociating the one or more particles comprising the unattached non-dissociable attachment handles from the solid support, attaching the unattached non-dissociable attachment handles of the one or more particles to complementary non-dissociable attachment handles of the solid support or a second solid support.

After removing one or more particles having an incomplete complement of attached entities, the particles may be provided to another process or assay, for example as reagents for the process or assay. In some cases, particles of an enriched particle mixture may be attached to a second solid support. For example, an enriched particle mixture comprising particles attached to analytes may be contacted to a solid support comprising an array of sites, thereby forming an array of analytes. In some cases, attaching particles of an enriched particle mixture to a second solid support can comprise: (i) providing the second solid support, in which the second solid support comprises a plurality of sites, in which each site of the plurality of sites is optically resolvable from any other site of the plurality of sites, and (ii) attaching particles of the particle mixture to the plurality of sites, in which each site of the plurality of sites is attached to only one particle of the particle mixture. Accordingly, useful molecules or moieties for anchoring moieties, as set forth herein, may be useful as particles of a particle mixture. In particular cases, after dissociating particles of a particle mixture from a solid support, each particle of the particle mixture may comprise a dissociable attachment handle or a plurality thereof. Accordingly, attaching the particle mixture to the second solid support can comprise attaching dissociable attachment handles of particles of the particle mixture to complementary dissociable attachment handles of the second solid support. For example, each site of a plurality of sites may comprise one or more complementary dissociable attachment handles. Accordingly, a complementary dissociable attachment handle of each array site may be bound to a dissociable attachment handle of a single particle of a particle mixture, thereby binding the only one particle to the site.

In another embodiment, each particle of an enriched particle mixture may comprise an attached affinity agent and/or detectable label. In some cases, particles of an enriched particle mixture may comprise a plurality of attached affinity agents and/or detectable labels. The particles of the particle mixture may be useful as a binding reagent for assays that utilize binding reagents, including various single-analyte assays set forth herein. Accordingly, attaching particles of the particle mixture to a second solid support may comprise binding affinity agents of particles of the particle mixture to analytes attached to the second solid support. In some cases, the second support may comprise an array of sites, in which a plurality of analytes is immobilized on the sites of the array of sites, in which each site individually comprises only analyte of the plurality of analytes. Particle of the enriched particle mixture may be bound to a subset of the analytes of the plurality of analytes.

Some methods and systems set forth herein utilize common attachment handles to facilitate attachment of particles to entities such as analytes, affinity reagents, oligonucleotides, tether strands, and/or docker strands. Attachment systems often comprise an attachment handle and a complementary attachment handle that are configured to form a covalent or non-covalent binding interaction. Examples of such systems can include oligonucleotide-oligonucleotide complexes, Click-type reactant pairs (e.g., mTz-TCO, DBCO-azide, etc.), non-covalent receptor-ligand binding pairs (e.g., streptavidin/biotin, neutravidin/biotin, etc.), covalent receptor-ligand binding pairs (e.g., SpyCatcher/SpyTag, SnoopCatchter/SnoopTag, SdyCatcher/SdyTag, etc.), and chelating complexes (e.g., Ni-NTA/polyhistidine). In some cases, free or unattached attachment handles may provide sufficient antigenicity for affinity reagents. Accordingly, it may be useful to provide affinity reagents to methods and systems of the present disclosure that comprise affinity reagents with binding specificities for attachment handles provided herein. The presence of unattached attachment handles may be useful for scavenging unattached particles from a heterogeneous particle mixture. For example, a particle comprising an unattached Click reagent (e.g., DBCO, TCO, etc.) due to not attaching to an mTz-modified entity may be bound to a solid support comprising an immobilized antibody with a binding specificity for the Click reagent. The presence of unattached attachment handles may be useful for scavenging complexed particles from a heterogeneous particle mixture. For example, a solid support comprising an immobilized antibody having a binding specificity for mTz may be bound to a particle-attached analyte that is modified with an excess of mTz.

Particle size or complexed particle size may create steric limitations that slow the rate of attachment of the particle to a solid support or inhibit the attachment of the particle to the solid support. For attachment via affinity reagents such as antibodies, binding of affinity reagents to an attachment handle of a particle or complexed particle may occur in a fluid phase, followed by attachment of the particle-affinity reagent complex or complexed particle-affinity reagent complex to a solid support. Solid supports comprising immobilized antibody-binding proteins (e.g., protein A, protein G, protein A/G, protein L, secondary antibodies, etc.) may be useful for attaching particles to a solid support. Affinity reagents can also be provided with an attachment handle (e.g., an attached oligonucleotide, peptide tag, binding ligand, chelating moiety, etc.) that can be attached to the solid support or a complementary attachment handle immobilized thereto. Alternatively, an attachment handle may be immobilized on a solid support by a flexible linker (e.g., a polymer strand) that can diffuse with some degrees of freedom in a fluid phase, thereby overcoming steric occlusion caused by particle or complexed particle size.

In some cases, an enriched particle mixture, as set forth herein, may be utilized for a

The present disclosure further provides systems for particle separation, comprising: (a) a vessel containing a fluid phase, wherein the fluid phase is contacted to a solid support, as set forth herein, within the vessel, (b) a particle mixture, as set forth herein, comprising a first set of particles and a second set of particles, (c) a particle transfer device, wherein the particle transfer device is configured to transfer the particle mixture to the vessel containing the fluid phase, and (d) a separation device, wherein the separation device is configured to separate the fluid phase from contact with the solid support.

A system may comprise a vessel that is configured to receive a fluid phase. Useful vessels may include tubes and reservoirs. A system may include a single vessel (e.g., an Eppendorf tube) or a plurality of vessels (e.g., a 96-, 192-, or 384-well plate). A vessel may be configured to attach to or fit with a separation device. For example, a vessel may have an appropriate size or shape that fits a centrifuge device. In another example, a vessel may have a shape or be provided with an attachment apparatus that facilitates assembly of the vessel with a magnetic or electrical separation device.

A system may further comprise a particle transfer device. A particle transfer device may be configured to transfer a fluid phase comprising particles and/or a solid support. A particle transfer device may also include a mixing device that facilitates contacting of a particle mixture with a solid support. Exemplary particle transfer devices can include pipettes, auto-pipetting devices, and decanters.

A system may further comprise a separation device. A separation device may be configured to separate a solid support from a fluid phase according to a method set forth herein. Exemplary separation devices can include centrifuges, sedimentation tubes, magnets, electromagnets, electrode devices, and combinations thereof.

The present disclosure also provides kits for particle separation. A kit may comprise a vessel. The vessel may be provided with a solid support, as set forth herein, contained within the vessel (e.g., a solid support in a fluid phase, a solid support in a powdered, precipitated, or crystallized form). Alternatively, a solid support may be provided in a kit in a separate vessel from the vessel that is configured to effect particle separation. A kit may further comprise particles. The particles may be provided in a fluid phase or in a solid phase. The particles may be attached to entities, as set forth herein. Alternatively, the particles may not be attached to entities. Accordingly, a kit may further comprise one or more reagents that are configured to facilitate formation of complexed particles, as set forth herein, or one or more reagents that are configured to facilitate a method of particle separation, as set forth herein.

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI. 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

A protein can be attached to a unique identifier using any of a variety of means. The attachment can be covalent or non-covalent. Exemplary covalent attachments include chemical linkers such as those achieved using click chemistry or other linkages known in the art or described in U.S. Pat. No. 11,203,612, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patent application Ser. No. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g. ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g. an address in an array) using any of a variety of means. The attachment can be covalent or non-covalent. Exemplary covalent attachments include chemical linkers such as those achieved using click chemistry or other linkages known in the art or described in U.S. Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in U.S. Pat. Nos. 11,203,612 or 11,505,796 or US Pat. App. Pub. No 2023/0167488 A1, each of which is incorporated herein by reference.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440: 297-302 (2006); Sigle et al, Nature Materials 20: 1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; or US Pat. App. Pub. No. 2022/0162684 A1 or 2023/0167488 A1, each of which is incorporated herein by reference.

A structured nucleic acid particle (e.g. having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g. protein), paratope, affinity moiety (e.g. antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440: 297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

An retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), peptides (e.g. proteins, polypeptides or oligopeptides), amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40: 2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17): 3982 (2020); Zheng, et al. or Chem. Soc. Rev. 44, 4091-4130 (2015), each of which is incorporated herein by reference.

The present disclosure provides compositions and methods for improving binding of analytes to affinity reagents by increasing avidity of the binding interaction. In particular embodiments, avidity between an analyte and affinity reagent can be increased by association of a docker with the analyte and association of a tether with the affinity reagent. The docker and tether recognize each other and can thus bind to each other. Avidity of the interaction between the affinity reagent and analyte is a function not only of recognition between the paratope and epitope, but also recognition between the docker and tether.

A docker can be associated with an analyte via covalent and/or non-covalent attachment of the docker to the analyte. Similarly, a tether can be associated with an affinity reagent via covalent and/or non-covalent attachment of the docker to the affinity reagent. Exemplary attachment chemistries include those set forth herein in the context of attaching analytes and affinity reagents to retaining components, addresses of an array, solid supports, labels, etc. In some configurations, a docker or tether can be attached to a particle (e.g. structured nucleic acid particle), unique identifier, address or solid support to which an analyte or affinity reagent, respectively, is attached.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes a step of detecting association of the affinity reagent with the analyte, thereby identifying the analyte. In another option, the analyte is present in a sample including other analytes and the method further includes a step of separating the analyte from the other analytes via the association of the affinity reagent with the analyte.

The compositions and methods of the present disclosure are particularly well suited for detecting analytes using affinity reagents in non-equilibrium conditions. A typical binding assay employ an excess amount of affinity reagent and immobilized analytes to drive formation of an immobilized complex between the affinity reagent and analyte. In some assays the excess labeled affinity reagent in solution produces unwanted background that overwhelms signal produced by immobilized complexes. Removal of excess affinity reagents from solution creates a non-equilibrium condition that drives affinity reagents to dissociate from the immobilized analytes. The use of tethers and dockers can increase the half-life of the complexes under non-equilibrium conditions, thereby improving detectability of analyte-affinity reagent complexes.

A variety of different types of dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker type and tether type.

A docker can be any molecule or moiety that is capable of binding to a tether and a tether can be any molecule or moiety that is capable of binding to a docker. A particularly useful docker or tether is a nucleic acid strand having a nucleotide sequence that complements a nucleotide sequences of a tether or docker, respectively. A nucleic acid strand that is used as a docker or tether can include a sequence of at least 3, 4, 5, 6, 7, 8, 9, 10, 12, 14, 16, 18, 20, 25 or more nucleotides. Alternatively or additionally, a nucleic acid strand that is used as a docker or tether can include a sequence of at most 25, 20, 18, 16, 14, 12, 10, 9, 8, 7, 6, 5, 4, 3 or fewer nucleotides. Other useful dockers or tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers or tethers include, but are not limited to, an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker or tether can be a protein that recognizes a nucleic acid sequence such as a DNA binding protein or RNA binding protein. Exemplary nucleic acid-binding proteins, which can be used as dockers or tethers, and the nucleic acid moieties to which they bind, which can be used as tethers or dockers, respectively, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See Leung et al. Nucleic Acids Res. 47 (Database issue): D322-D329 (2019), which is incorporated herein by reference.

A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope.

Of course, a binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tm) of the duplex formed by the complementary sequences. The length of the complementary sequences can be at least 3, 4, 5, 6, 7, 8, 9, 10, 12, 14, 16, 18, 20, 25 or more nucleotides. Alternatively or additionally, the length of the complementary sequences can be at most 25, 20, 18, 16, 14, 12, 10, 9, 8, 7, 6, 5, 4, 3 or fewer nucleotides. The GC content of the complementary sequences can be at least 25%, 40%, 50%, 60%, 75%, or higher. Alternatively or additionally, the GC content of the complementary sequences can be at most 75%, 60%, 50%, 40%, 25% or lower.

Multiplex methods, in which a plurality of different analytes are processed in parallel, can employ universal dockers. The dockers are referred to as ‘universal’ because they are identical with respect to structural features that interact with tethers. For example, an array can include a plurality of addresses, each of the addresses being attached to an analyte that differs from other analytes in the array and each of the addresses being attached to a docker that is the same as other dockers in the array. A plurality of different analytes that are associated with universal dockers can be contacted with a plurality of different affinity reagents that are associated with tethers. Some or all the different affinity reagents can have the same tether structure. As such, the avidity effect of the dockers and tethers can be substantially uniform.

Methods that employ multiple different affinity reagents can employ universal tethers. The tethers are referred to as ‘universal’ because they are identical with respect to structural features that interact with dockers. For example, an array of analytes can be contacted with a plurality of different affinity reagents, each of the affinity reagents having a paratope that differs from other affinity reagents in the plurality and each of the affinity reagents being attached to a tether that is the same as other tethers in the plurality. The different affinity reagents can be present in a mixture that is simultaneously in contact with the array or, alternatively, the different affinity reagents can be serially contacted with the array.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)—tetracyclooctylene (TCO), azide—dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13: 10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14: 206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1: 845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PUP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27: 953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylglucosaminidase can be used to cleave non-reducing terminal β-linked N-acetylglucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

In addition to the foregoing reagents, also provided herein are kits useful in carrying out some methods described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

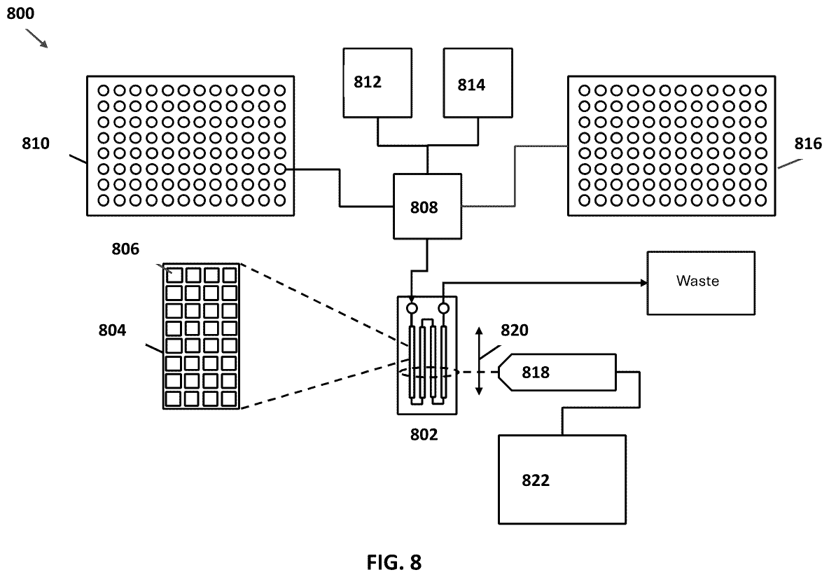

Additionally, provided herein are systems for performing assays on enriched particle mixtures, as set forth herein, by methods described herein. An example of a system is illustrated in FIG. 8. As shown, the system 800 includes a flowcell 802 that includes an array surface (shown as 804) within the channels of the flow cell upon which individual analyte molecules from a sample may be deposited and immobilized in locations 806 that are individually addressable, and in particular cases are individually optically resolvable from each other using, e.g., fluorescence microscopy or scanning techniques.

The system will also typically include a fluidic delivery system 808 that is configured to deliver different fluids to the flow cell 802 through a series of fluidic lines and utilizing appropriate pumps, valves and other conventional fluid controls. The fluidics system 808 may be fluidically coupled to various sources of fluids and reagents needed to carry out the analysis on the flow cell. For example, as shown, fluidic system 808 is fluidly coupled to a source of a plurality of reagents 810 (shown as a 96 well plate, although any number of different reagent storage systems of varying capacity may be employed) that includes a library of multiple affinity reagents that each have affinity for different characteristics of one or more proteins of interest. Additionally, fluidic system 808 may also be coupled to sources of washing fluids or buffers 812, and removal reagents 814 (for removing bound affinity reagents following detection), as well as any other ancillary fluids and reagents needed for the analysis. Similarly, where flow cells are prepared on the system, the fluidic system may be coupled to sources of different sample materials that are to be analyzed 816 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 818, for detecting and recording fluorescent signals arising from different positions on the array surface. Such detection systems may generally include line scanning confocal fluorescent microscope systems, which are capable of scanning across large array surfaces (as shown by arrow 820) to detect and record fluorescence across such surfaces at reasonably high scan rates.

The overall systems also typically include one or more computers or processors 822 for controlling the operation of the instrument system including the fluidic system 808 (e.g., to sample different sample sources 816, reagent sources 810 and delivery timing and volume of each), and detection system 818, among other functions, and for recording the detected signals received from the detection system 818, e.g. fluorescent signals, and analyzing such signals to identify potential binding by each of the different affinity reagents. Processors 822 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, bioRxiv, https://doi.org/10.1101/2021.10.11.463967, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 822 can be an electronic device of a detection system, the electronic device being integral to the detection system or remotely located with respect to the detection system. The computer system 822 includes a computer processing unit (CPU, also “processor” and “computer processor” herein), which can be a single core or multi core processor, or a plurality of processors for parallel processing. The computer system 822 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 822 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 822, can implement a peer-to-peer network, which may enable devices coupled to the computer system 822 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 822 can be included in the circuit. In some cases, the circuit is an application specific integrated circuit (ASIC).

The storage unit can store files, such as drivers, libraries and saved programs. The storage unit can store user data, e.g., user preferences and user programs. The computer system 822 in some cases can include one or more additional data storage units that are external to the computer system 822, such as located on a remote server that is in communication with the computer system 822 through an intranet or the Internet.

The computer system 822 can communicate with one or more remote computer systems through the network. For instance, the computer system 822 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 822 via the network.

Methods as described herein can be implemented by way of machine (e.g., computer processor) executable code stored on an electronic storage location of the computer system 822, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 822, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (TR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 822 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### EXAMPLES

### Example 1. Purification of Analyte-Coupled Particles

Protein analytes coupled to nucleic acid nanoparticles were purified from crude samples comprising unattached proteins, unattached nucleic acid nanoparticles, and defective nucleic acid nanoparticles via capture on magnetic AMPure beads. Crude mixtures containing nucleic acid nanoparticles attached to protein analytes were prepared by methods set forth in U.S. Pat. No. 11,505,796. Protein analytes modified with methyltetrazine (mTz) functional groups were attached to nucleic acid nanoparticles, with each nanoparticle having a single trans-cyclooctene (TCO) functional group for attaching a single protein analyte. The oligonucleotide that bound the TCO functional group to a nucleic acid nanoparticle contained a single-stranded capture sequence that could facilitate capture on AMPure beads

AMPure beads were conjugated to oligonucleotides with a nucleotide sequence complementary to the capture sequence of the nucleic acid nanoparticles, as well as methyltetrazine functional groups. AMPure beads were prepared by combining the AMPure beads as provided by the vendor with 200 mM magnesium chloride solution until the final concentration of MgCl₂ was 12.5 mM. Beads were mixed to ensure suspension and equilibrated to room temperature before use.

50 microliter (μL) of crude particle mixture was combined with 30 μL of prepared AMPure beads. Beads and particles were incubated at room temperature for 5 minutes with shaking at 600 rpm. After incubation and mixing, particles were placed in a magnetic separator for 2 minutes to separate the beads from the fluid phase. After magnetic separation, the supernatant was pipetted from the tube containing the AMPure beads. With the tube containing the separated beads still contacted to the magnet of the magnetic separator, 180 μL of 75% ethanol was added to the tube and incubated for 5 seconds. The ethanol was removed from the tube, then the tube was removed from the magnetic separator.

50 μL of magnesium chloride-containing buffer was added to the tube to elute the analyte-attached nucleic acid nanoparticles. Particles were eluted for 5 minutes at room temperature with 600 rpm shaking. After elution, the tube was returned to the magnetic separator for 2 minutes. The fluid was extracted from the tube without disturbing the pelletized AMPure beads, thereby obtaining a particle mixture enriched for particles attached to protein analytes.

### Example 2. Purification of Analyte-Coupled Particles with Secondary Selection

After performing the method described in Example 1, the enriched particle mixture is contacted with a second fraction of AMPure beads. The AMPure beads are attached to oligonucleotide having a same sequence as the oligonucleotide that attaches TCO functional groups to nucleic acid nanoparticles. 50 μL of particle mixture is mixed with 30 μL of AMPure beads. Beads and particles are incubated at 30° C. for 30 minutes with shaking at 600 rpm. After incubation and mixing, particles are placed in a magnetic separator for 2 minutes to separate the beads from the fluid phase. The fluid is extracted from the tube without disturbing the pelletized AMPure beads, thereby obtaining a particle mixture depleted for particles lacking a TCO-coupled oligonucleotide.

### Example 3. Formation of a Heterogenous Particle Mixture

A plurality of different polypeptides is modified to attach methyltetrazine (mTz) molecules to thiol groups of cysteine sidechains of the polypeptides. Polypeptides have a dispersity of total attached mTz molecules depending upon the total quantity of cysteine residues in each protein molecules and the overall extent of functionalization. After mTz modification, the plurality of polypeptides is incubated with a plurality of particles. Each particle of the plurality of particles is attached to only one trans-cyclooctene (TCO) molecule. Particles of the plurality of particles are attached to polypeptides of the polypeptide mixture by reaction of a methyltetrazine moieties of polypeptides with TCO moieties of particles. A heterogeneous particle mixture is formed, comprising unattached particles having unattached TCO moieties, particles attached to single polypeptides, and unattached polypeptides. Unattached proteins are separated from the heterogeneous particle mixture by size exclusion chromatography, with the particle-rich fraction retained after chromatographic separation.

### Example 4. Modification of Analyte-Coupled Particles with Peptide Tags

The heterogenous particle mixture of Example 3 is incubated with TCO-terminated peptide tags comprising a 12 amino acid polyhistidine sequence. The TCO-terminated peptide tags attach to unbound mTz molecules of polypeptides, thereby providing peptide tags to particle-attached polypeptides of the heterogeneous particle mixture. The quantity of peptide tags present on particles of the heterogeneous particle mixture varies from zero to two or more depending upon the number of available mTz moieties on each individual polypeptide.

### Example 5. Separation of Analyte-Coupled Particles with Antibodies

The heterogeneous particle mixture of Example 4 is incubated in a vessel with a solid support comprising magnetic beads attached to anti-polyhistidine antibodies. Particles are attached to the solid support by binding of anti-polyhistidine antibodies to peptide-tagged polypeptides. After incubating the heterogeneous particle mixture with the solid support, the solid support is collected on a surface of the vessel using a magnet. The liquid comprising unbound particles is decanted from the vessel. After decanting, the vessel is filled with a fluid phase comprising guanidinium hydrochloride and shaken, thereby eluting the particles from the antibodies of the solid support. The solid support is again separated from the fluid phase by applying a magnetic field to the vessel. The fluid phase containing the polypeptide-attached particles is decanted from the vessel, thereby providing an enriched particle mixture containing single particles attached to only one polypeptide.

### Example 6. Separation of Analyte-Coupled Particles with Chelation

The heterogeneous particle mixture of Example 4 is incubated in a vessel with a solid support comprising magnetic beads attached to nickel nitrilotriacetic acid moieties (Ni-NTA). Particles are attached to the solid support by binding of Ni-NTA to polyhistidine sequences of the peptide-tagged polypeptides. After incubating the heterogeneous particle mixture with the solid support, the solid support is collected on a surface of the vessel using a magnet. The liquid comprising unbound particles is decanted from the vessel. After decanting, the vessel is filled with a fluid phase comprising histidine and shaken, thereby eluting the particles from the antibodies of the solid support. The solid support is again separated from the fluid phase by applying a magnetic field to the vessel. The fluid phase containing the polypeptide-attached particles is decanted from the vessel, thereby providing an enriched particle mixture containing single particles attached to only one polypeptide.

### Example 7. Separation of Analyte-Coupled Particles with Antibodies

The heterogeneous particle mixture of Example 3 is incubated in a vessel with a solid support comprising magnetic beads attached to antibodies having a binding specificity to TCO moieties. Particles are attached to the solid support by binding of antibodies to TCO moieties of the particles not attached to polypeptides. After incubating the heterogeneous particle mixture with the solid support, the solid support is collected on a surface of the vessel using a magnet. The liquid comprising polypeptide-attached particles is decanted from the vessel, thereby providing an enriched particle mixture containing single particles attached to only one polypeptide.

### Example 8. Separation of Analyte-Coupled Particles with Antibodies

The heterogeneous particle mixture of Example 3 is incubated in a vessel with fluid phase antibodies having a binding specificity to mTz moieties. Antibodies are incubated with particles for an hour. After incubation, the heterogeneous particle mixture is contacted to a solid support comprising immobilized protein A molecules. The protein A molecules bind antibodies, thereby attaching particles to the solid support. After incubating the antibodies with the solid support, the solid support is collected on a surface of the vessel using a magnet. The liquid comprising unbound particles is decanted from the vessel. After decanting, the vessel is filled with a fluid phase comprising glycine hydrochloride, pH 2.5, and shaken, thereby eluting the particles from the solid support. The solid support is again separated from the fluid phase by applying a magnetic field to the vessel. The fluid phase containing the polypeptide-attached particles is decanted from the vessel, thereby providing an enriched particle mixture containing single particles attached to only one polypeptide.

### Example 9. Separation of Analyte-Coupled Particles with Antibodies

Polypeptides derived from a biological sample are attached to particles according to the method of Example 3 to form a heterogeneous particle mixture. The polypeptides contain a plurality of different naturally-occurring post-translational modifications (PTMs) to C-terminal and/or N-terminal amino acids. Common naturally-occurring terminal PTMs (e.g., acetylation, methylation, glycosylation, ubiquitination, etc.) are described in Chen and Kashina, Front Cell Dev Biol, (2021), which is herein incorporated by reference in its entirety.

The heterogeneous particle mixture is incubated with a solid support comprising a plurality of beads. Beads of the plurality of beads are attached to antibodies having binding specificities for differing terminal PTMs. Each bead contains a single type of antibody, each antibody immobilized to a bead having the same structure. Antibodies of beads bind to terminal PTMs of polypeptides attached to particles, thereby binding particles of the heterogeneous particle mixture to the solid support. After incubating the heterogeneous particle mixture with the solid support, the solid support is collected on a surface of the vessel using a magnet. The liquid comprising unbound particles is decanted from the vessel. After decanting, the vessel is filled with a fluid phase comprising guanidinium hydrochloride and shaken, thereby eluting the particles from the antibodies of the solid support. The solid support is again separated from the fluid phase by applying a magnetic field to the vessel. The fluid phase containing the polypeptide-attached particles is decanted from the vessel, thereby providing an enriched particle mixture containing single particles attached to only one polypeptide.

### Example 10. Screening of Antibody Affinity Reagent for Click Reactant

Antibodies that recognize the methyltetrazine (mTz) click reactant in its unreacted form were identified by a phage display process. A phage display library (ALTHEA Gold; Antibody Design Labs, San Diego, CA) of scFv binders was panned against peptide targets containing mTz-modified amino acids. Table I lists the peptide targets utilized for the panning stages. Each peptide target contained a lysine residue (K). The lysine of each peptide target was modified to attach to an mTz functional group by reacting the lysine's sidechain amine functional group with an NHS-(PEG)_{˜}-mTz molecule. Modified lysine residues are denoted in Table I with an asterisk (K*). Each peptide target was flanked on its N-terminal and C-terminal sides by a non-antigenic linker peptide having an amino acid sequence GGAAGG. Each peptide contained a terminal biotin that was used to attach the peptide targets to streptavidin-coated beads.

TABLE I

| Target # | Peptide Sequence |
|---|---|
| 1 | K*NF |
| 2 | IFK* |
| 3 | DFK* |
| 4 | YK*E |
| 5 | FSK* |
| 6 | K*YQ |

The phage display library was individually screened against each peptide target. After panning, selected clones were amplified by transduction into bacterial cells. In total, 22 clones were panned from amongst the 6 unique peptide targets listed in Table I. After panning, binding specificity of each clone was confirmed by a phage ELISA assay, utilizing the original target for each clone as the positive control and a version of the target with an unmodified lysine as the negative control. 22 clones were selected for further analysis. Amplified clones from each panning process were screened against a wider panel of peptide targets via phage ELISA to determine binding specificity. Phage ELISA screenings for each selected phage included screening a phage against its panning target, screening the phage against different targets containing mTz-modified peptides, and screening the phage against targets containing unmodified lysine residues (KNF, IFK, DFK, YKE, FSK, KYQ). Table II lists the results for Phage ELISA for each panned phage. X marks indicate an observed presence of binding of the scFv to the screening peptide target. The rightmost column lists any unmodified peptides that were bound by the scFv. 19 scFv fragments were identified that bound to all peptide targets containing an mTz moiety but did not bind to any targets containing unmodified lysines, thereby suggesting that the scFv were mTz-specific.

TABLE II

| | Panning | | | | | | | |
|---|---|---|---|---|---|---|---|---|
| | Target | Screening Target Peptide | | | | | | |
| Pan # | Peptide | K*NF | FSK* | DFK* | IFK* | YK*E | K*YQ | Unmod. |
| 1 | K*NF | X | X | X | X | X | X | — |
| 2 | IFK* | X | X | X | X | X | X | — |
| 3 | K*NF | X | X | X | X | X | X | — |
| 4 | DFK* | X | X | X | X | X | X | — |
| 5 | YK*E | X | X | X | X | X | X | — |
| 6 | K*NF | X | X | X | X | X | X | — |
| 7 | FSK* | X | X | X | X | X | X | — |
| 8 | FSK* | X | X | X | X | X | X | — |
| 9 | K*YQ | X | X | X | X | X | X | — |
| 10 | FSK* | X | X | X | X | X | X | — |
| 11 | FSK* | X | X | X | X | X | X | — |
| 12 | DFK* | X | X | X | X | X | X | — |
| 13 | IFK* | X | X | X | X | X | X | — |
| 14 | IFK* | — | — | — | X | — | — | IFK |
| 15 | IFK* | X | X | X | X | X | X | — |
| 16 | IFK* | X | X | X | X | X | X | — |
| 17 | IFK* | X | X | X | X | X | X | — |
| 18 | IFK* | — | X | X | X | X | — | — |
| 19 | YK*E | — | — | — | — | X | — | — |
| 20 | YK*E | X | X | X | X | X | X | — |
| 21 | YK*E | X | X | X | X | X | X | — |
| 22 | K*YQ | X | X | X | X | X | X | — |

Each of the 19 identified phage had their genomes sequenced to identify the amino acid sequence of their respective complementarity-determining regions (CDRs). 5 of the panned phages were determined to be duplicates (i.e., phages displaying the same scFv that bound to different peptide targets). Accordingly, 14 unique mTz-binding scFvs were identified by the panning processes. Table III lists heavy chain (HC) and light chain (LC) sequences for each of the 14 identified scFvs. The light chain and heavy chain sequences represented by SEQ TD NOS: 1-2, 3-4, and 9-10 were converted into IgG antibodies by inserting them into a human IgG1 scaffold.

TABLE III

| | Light Chain | | Heavy Chain | |
|---|---|---|---|---|
| Clone | SEQ. | | SEQ. | |
| No. | ID | AA Sequence | ID | AA Sequence |
| 1 | 1 | DIVMTQSPDSLAVSL | 2 | EVQLLESGGGLVQPGGSLRLSCAAS |
| | | GERATINCKSSQSVL | | GFTFSSYAMSWVRQAPGKGLEWVS |
| | | RSSNNENYLAWYQQ | | GISGYGGDTDYADSVKGRFTISRDN |
| | | KPGQPPKLLIYGAST | | SKNTLYLQMNSLRAEDTAVYYCAG |
| | | RESGVPDRFSGSGSG | | PGYCSGGSCYRWGQGTLVTVSS |
| | | TDFTLTISSLQAEDV | | |
| | | AVYYCQQWYSAPW | | |
| | | TFGQGTKVEIK | | |
| 2 | 3 | DIVMTQSPDSLAVSL | 4 | EVQLLESGGGLVQPGGSLRLSCAAS |
| | | GERATINCKSSQSVL | | GFTFSSYAMSWVRQAPGKGLEWVS |
| | | YSSNNKNYLAWYQQ | | AISGSGYGGDTYYADSVKGRFTISR |
| | | KPGQPPKLLIYAAST | | DNSKNTLYLQMNSLRAEDTAVYYC |
| | | RESGVPDRFSGSGSG | | AGPGYCSGGSCYRWGQGTLVTVSS |
| | | TDFTLTISSLQAEDV | | |
| | | AVYYCQQWYRAPYT | | |
| | | FGQGTKVEIK | | |
| 3 | 5 | EIVLTQSPGTLSLSPG | 6 | EVQLLESGGGLVQPGGSLRLSCAAS |
| | | ERATLSCRASQSVSS | | GFTFTNYAMSWVRQAPGKGLEWVS |
| | | SYLAWYQQKPGQAP | | AISGSGGSTSYADSVKGRFTISRDNS |
| | | RLLIYGASSRATGIPD | | KNTLYLQMNSLRAEDTAVYYCAR |
| | | RFSGSGSGTDFTLTIS | | WDYWGQGTLVTVSS |
| | | RLEPEDFAVYYCQQ | | |
| | | HESWPITFGQGTKVE | | |
| | | IK | | |
| 4 | 7 | DIVMTQSPDSLAVSL | 8 | EVQLLESGGGLVQPGGSLRLSCAAS |
| | | GERATINCKSSQSVL | | GFTFTDYQMSWVRQAPGKGLEWVS |
| | | YSSNNENYLAWYQQ | | AIDGSDTYYADSVKGRFTISRDNSK |
| | | KPGQPPKLLIYGAST | | NTMYLQMNSLRAEDKAVYYCARA |
| | | RESGVPDRFSGSGSG | | PSGWAPGYFDYWGQGTLVTVSS |
| | | TDFTLTISSLQAEDV | | |
| | | AVYYCQQGSNAPYT | | |
| | | FGQGTKVEIK | | |
| 5 | 9 | DIVMTQSPDSLAVSL | 10 | EVQLLESGGGLVQPGGSLRLSCAAS |
| | | GERATINCKSSQSVL | | GFTFTDYQMSWVRQAPGKGLEWVS |
| | | YSSNNENYLAWYQQ | | AIDGSDTYYADSVKGRFTISRDNSK |
| | | KPGQPPKLLIYGAST | | NTLYLQMNSLRAEDTAVYYCARAS |
| | | RESGVPDRFSGSGSG | | SGWAPGYFDYWGQGTLVTVSS |
| | | TDFTLTISSLQAEDV | | |
| | | AVYYCQQGSNAPYT | | |
| | | FGQGTKVEIK | | |
| 6 | 11 | DIVMTQSPDSLAVSL | 12 | EVQLLESGGGLVQPGGSLRLSCAAS |
| | | GERATINCKSSQSVL | | GFTFSDYAMSWVRQAPGKGLEWVS |
| | | RSNNNENYLAWYQQ | | AISGGGGYTYYADSVKGRFTISRDN |
| | | KPGQPPKLLIYGAST | | SKNTLYLQMNSLRAEDTAVYYCAK |
| | | RESGVPDRFSGSGSG | | NRCSDYCIPDWFDPWGQGTLVTVSS |
| | | TDFTLTISSLQAEDV | | |
| | | AVYYCQQGYRYPLT | | |
| | | FGQGTKVEIK | | |
| 7 | 13 | DIVMTQSPDSLAVSL | 14 | EVQLLESGGGLVQPGGSLRLSCAAS |
| | | GERATINCKSSQSVL | | GFTFTDDAMSWVRQAPAKGLEWVS |
| | | YSDNNENYLAWYQQ | | AISGRGGTTNYADSVKGRFTISRDNS |
| | | KPGQPPKLLIYWAST | | NNTLYLQMNSLRAEDTAVYYCAGP |
| | | RESGVPDRFSGSGSG | | GYCSGGSCYRWGQGTLVTVSS |
| | | TDFTLTISSLQAEDV | | |
| | | AVYYCQQWYSEPFT | | |
| | | FGQGTKVEIK | | |
| 8 | 15 | EIVLTQSPGTLSLSPG | 16 | EVQLLESGGGLVQPGGSLRLSCAAS |
| | | ERATLSCRASQSVSA | | GFTFTSYGMSWVRQAPGKGLEWVS |
| | | SYLAWYQQKPGQAP | | GISGSGYTTSYADSVKGRFTISRDNS |
| | | RLLIYGASSRATGIPD | | KNTLYLQMNSLRAEDTAVYYCASG |
| | | RFSGSGSGTDFTLTIS | | GYWGQGTLVTVSS |
| | | RLEPEDFAVYYCQQ | | |
| | | YSSYPFTFGQGTKVE | | |
| | | IK | | |
| 9 | 17 | EIVLTQSPGTLSLSPG | 18 | EVQLLESGGGLVQPGGSLRLSCAAS |
| | | ERATLSCRASQSVSS | | GFTFTDSGMSWVRQAPGKGLEWVS |
| | | SALAWYQQKPGQAP | | RISGRGGTTDYADSVKGRFTISRDNS |
| | | RLLIYGASSRATGIPD | | KNTLYLQMNSLRAEDTAVYYCATD |
| | | RFSGSGSGTDFTLTIS | | PVFGYSSGWRDYWGQGTL VTVSS |
| | | RLEPEDFAVYYCQQ | | |
| | | HGSSPYTFGQGTKVE | | |
| | | IK | | |
| 10 | 19 | EIVLTQSPGTLSLSPG | 20 | EVQLLESGGGLVQPGGSLRLSCAAS |
| | | ERATLSCRASQSVSA | | GFTFSDSGMSWVRQAPGKGLEWVS |
| | | SNLAWYQQKPGQAP | | RIYGSDTYYADSVKGRFTISRDNSK |
| | | RLLIYAASSRATGIPD | | NTLYLQMNSLRAEDTAVYYCATDP |
| | | RFSGSGSGTDFTLTIS | | FYSYVDYWGQGTLVTVSS |
| | | RLEPEDFAVYYCQQ | | |
| | | HGNSPFTFGQGTKVE | | |
| | | IK | | |
| 11 | 21 | DIVMTQSPDSLAVSL | 22 | EVQLLESGGGLVQPGGSLRLSCAAS |
| | | GERATINCKSSQSVL | | GFTFSSYAMSWVRQAPGKGLEWVS |
| | | YSDNNENRLAWYQQ | | AISGSGYGGSTSYADSVKGRFTISRD |
| | | KPGQPPKLLIYAAST | | NSKNTLYLQMNSLRAEDTAVYYCA |
| | | RESGVPDRFSGSGSG | | GPGYCSGGSCYRWGQGTLVTVSS |
| | | TDFTLTISSLQAEDV | | |
| | | AVYYCQQWTSAPYT | | |
| | | FGQGTKVEIK | | |
| 12 | 23 | DIVMTQSPDSLAVSL | 24 | EVQLLESGGGLVQPGGSLRLSCAAS |
| | | GERATINCKSSQSVL | | GFTFSSYAMSWVRQAPGKGLEWVS |
| | | YSGNNKNYLAWYQ | | AISGSGGSTYYADSVKGRFTISRDNS |
| | | QKPGQPPKLLIYGAS | | KNTLYLQMNSLRAEDTAVYYCARH |
| | | TRESGVPDRFSGSGS | | WTYYFGSRSYSGAFDIWGQGTMVT |
| | | GTDFTLTISSLQAED | | VSS |
| | | VAVYYCQQYYNSPIT | | |
| | | FGQGTKVEIK | | |
| 13 | 25 | DIVMTQSPDSLAVSL | 26 | EVQLLESGGGLVQPGGSLRLSCAAS |
| | | GERATINCKSSQSVL | | GFTFSDYGMSWVRQAPGKGLEWVS |
| | | YSSNNENRLAWYQQ | | GISGRGGGTRYADSVKGRFTISRDN |
| | | KPGQPPKLLIYGAST | | SKNTLYLQMNSLRAEDTAVYYCAR |
| | | RESGVPDRFSGSGSG | | GVTHCTNGVCHYWYFDLWGRGTL |
| | | TDFTLTISSLQAEDV | | VTVSS |
| | | AVYYCQQYSNSPITF | | |
| | | GQGTKVEIK | | |
| 14 | 27 | EIVLTQSPGTLSLSPG | 28 | EVQLLESGGGLVQPGGSLRLSCAAS |
| | | ERATLSCRASQSVSS | | GFTFSSYGMSWVRQAPGKGLEWVS |
| | | SALAWYQQKPGQAP | | RISGSGDGSYTNYADSVKGRFTISRD |
| | | RLLIYGASSRATGIPD | | NSKNTLYLQMNSLRAEDTAVYYCA |
| | | RFSGSGSGTDFTLTIS | | SDPITMIVGDAFDIWGQGTMVTVSS |
| | | RLEPEDFAVYYCQQ | | |
| | | HSSSPFTFGQGTKVEI | | |
| | | K | | |

### Example 11. Separation of Complexed Particles

Complexed particles were separated from a mixture comprising a mixture of complexed particles and precursor entities. Each complexed particle comprised a single polypeptide attached to a single structured nucleic acid particle. Methods of forming complexed particles comprising a single polypeptide attached to a single particle are provided in U.S. Pat. Nos. 11,203,612 and 11,505,796, each of which is incorporated by reference. Prior to attachment to nucleic acid particles, the polypeptides were functionalized with methyltetrazine (mTz) moieties via modification of lysine sidechains. Each nucleic acid particle contained a single trans-cyclooctene molecule that reacted with an mTz moiety of a polypeptide, thereby attaching a single polypeptide to the nucleic acid particle. Nucleic acid particles also further contained Alexa Fluor 488 fluorescent dye molecules. Each polypeptide contained excess mTz moieties that were not attached to a nucleic acid particle. The mixture further comprised nucleic acid particles that were not attached to a polypeptide. The measured amount of complexed particle relative to total nucleic acid particles in the mixture was 31%.

The mixture of complexed particles and unattached nucleic acid particles was mixed with an IgG antibody with a binding specificity for mTz in a well of a well plate. The IgG antibody contained a light chain containing the amino acid sequence of SEQ ID NO: 3, and further contained a heavy chain containing the amino acid sequence of SEQ ID NO: 4. A first well contained antibody at an estimated 500× excess relative to expected quantity of complexed particles. The second well contained antibody at an estimated 50× excess relative to expected quantity of complexed particles. The fluid phase antibody was incubated with the mixture for 1 hour. After incubation, the mixture was contacted with magnetic beads attached to protein A molecules. The magnetic beads were placed in a magnet rack to draw the beads to the bottom of the well. After separating the magnetic beads, the supernatant was removed, thereby removing any entities not attached to the magnetic beads. The empty well was removed from the magnet rack, then the beads were resuspended in phosphate buffer solution.

The concentration of nucleic acid particles was measured by fluorescence detection on a fluorescent plate reader. Fluorescent particle standards were used to generate a calibration curve that facilitated correlation of fluorescence measurements to particle concentration. For the 500× antibody excess well, 26% of total particles were associated to beads. For the 50× antibody excess well, 33% of total particles were associated to beads. The measurements suggest that most complexed particles were attached to the beads by association of the mTz-binding antibodies to the protein A-coated beads.

Elution of complexed particles from beads was tested. The bead-attached complexed particles were incubated for 10 minutes in a buffer containing 5.2M guanidinium hydrochloride, 20 mM magnesium chloride, and 20 mM sodium acetate. After incubation, beads were separated from the solution containing the eluted complexed particles. The concentration of particles in the elution buffer and the concentration of particles in the bead fraction was measured. The bead fraction was determined to contain 3% of particles after elution. The elution method was repeated with an incubation time of 30 minutes. After 30 minutes incubation, the fraction of particle contained in the bead fraction was reduced to 2%, suggesting that most elution occurred within the first 10 minutes.

1. A method for separating particles, comprising:
(a) contacting in a fluid phase a plurality of particles to a solid support, wherein the plurality of particles comprises:
(i) a first set of particles, wherein each particle of the first set of particles comprises a first attachment handle; and
(ii) a second set of particles, wherein each particle of the second set of particles comprises a second attachment handle, and wherein each particle of the second set of particles is attached to an entity;
(b) attaching first attachment handles of particles of the first set of particles or second attachment handles of particles of the second set of particles to the solid support; and
(c) after attaching the first set of particles or particles of the second set of particles to the solid support, separating the first set of particles from the second set of particles.

2. The method of claim 1, further comprising attaching entities to particles, thereby forming the second set of particles.

3. The method of claim 2, wherein an entity of the entities comprises a plurality of second attachment handles.

4. The method of claim 3, wherein attaching entities to particles comprises attaching a second attachment handle of the plurality of second attachment handles of the entity to a first attachment handle of a particle of the particles.

5. The method of claim 2, wherein attaching entities to particles comprises attaching a first coupling moiety of a particle of the particles to a complementary coupling moiety of an entity of the entities.

6. The method of claim 5, wherein a first attachment handle is attached to the coupling moiety of the particle of the particles, and wherein attaching the first coupling moiety of the particle of the particles to the complementary coupling moiety of the entity of the entities further comprises dissociating the first attachment handle from the coupling moiety of the particle of the particles.

7. The method of claim 1, wherein separating the first set of particles from the second set of particles comprises separating the fluid phase from the solid support.

8. The method of claim 7, further comprising, after separating the fluid phase from the solid support, dissociating particles from the solid support.

9. The method of claim 1, wherein attaching first attachment handles of particles of the first set of particles or second attachment handles of particles of the second set of particles to the solid support comprises attaching first attachment handles of particles of the first set of particles to complementary attachment handles of the solid support.

10. The method of claim 9, wherein attaching first attachment handles of particles of the first set of particles to the complementary attachment handles of the solid support comprises forming dissociable binding interactions between first attachment handles of particles of the first set of particles and the complementary attachment handles of the solid support.

11. The method of claim 9, wherein attaching first attachment handles of particles of the first set of particles to the complementary attachment handles of the solid support comprises forming non-dissociable binding interactions between first attachment handles of particles of the first set of particles and the complementary attachment handles of the solid support.

12. The method of claim 1, wherein attaching first attachment handles of particles of the first set of particles or second attachment handles of particles of the second set of particles to the solid support comprises attaching first attachment handles of particles of the first set of particles and second attachment handles of particles of the second set of particles to complementary attachment handles of the solid support.

13. The method of claim 12, wherein attaching first attachment handles of particles of the first set of particles and second attachment handles of particles of the second set of particles to complementary attachment handles of the solid support comprises forming non-dissociable binding interactions between first attachment handles of the particles of the first set of particles and the complementary attachment handles of the solid support, and further comprises forming dissociable binding interactions between second attachment handles of the particles of the second set of particles and the complementary attachment handles of the solid support.

14. The method of claim 1, wherein attaching first attachment handles of particles of the first set of particles or second attachment handles of particles of the second set of particles to the solid support comprises coupling the first attachment handles or the second attachment handles to affinity reagents.

15. The method of claim 14, wherein the affinity reagents are immobilized on the solid support.

16. The method of claim 14, wherein coupling the first attachment handles or the second attachment handles to affinity reagents comprises incubating the affinity reagents with the first attachment handles or the second attachment handles in the fluid phase.

17. The method of claim 16, further comprising, after incubating the affinity reagents with the first attachment handles or the second attachment handles in the fluid phase, attaching the affinity reagents bound to first attachment handles or second attachment handles to the solid support.

18. The method of claim 17, wherein attaching the affinity reagents bound to first attachment handles or second attachment handles to the solid support comprises attaching a third attachment handle of an affinity reagent to a complementary attachment handle of the solid support.

19. A method for separating particles, comprising:
(a) contacting in a fluid phase a plurality of particles to a solid support, wherein the solid support comprises a plurality of complementary attachment handles, and wherein the plurality of particles comprises:
(i) a first set of particles, wherein the first set of particles is attached to a plurality of polypeptides, wherein each polypeptide of the plurality of polypeptides comprises a plurality of attachment handles; and
(ii) a second set of particles, wherein each particle of the second set of particles is not attached to a polypeptide;
(b) attaching attachment handles of polypeptides to the plurality of complementary attachment handles of the solid support, thereby attaching particles of the first set of particles to the solid support; and
(c) after attaching particles of the first set of particles to the solid support, separating the particles of the first set of particles from the second set of particles.

20.-22. (canceled)

23. A method for separating particles, comprising:
(a) contacting in a fluid phase a plurality of particles to a solid support, wherein the solid support comprises a plurality of complementary first attachment handles, and wherein the plurality of particles comprises:
(i) a first set of particles, wherein the first set of particles is attached to a plurality of polypeptides, wherein each polypeptide of the plurality of polypeptides is attached to a particle of the first set of particles by coupling of a second attachment handle of the polypeptide to a complementary second attachment handle of the particle; and
(ii) a second set of particles, wherein each particle of the second set of particles is not attached to a polypeptide, and wherein each particle of the second set of particles comprising an unbound complementary second attachment handle;
(b) attaching unbound complementary second attachment handles of particles of the second set of particles to the plurality of complementary attachment handles of the solid support, thereby attaching particles of the second set of particles to the solid support; and
(c) after attaching particles of the second set of particles to the solid support, separating the particles of the first set of particles from the second set of particles.

24.-27. (canceled)
